(19) United States
(12) Patent Application Publication
(10) Pub. No.: US 2008/0057494 A1
(43) Pub. Date: Mar. 6, 2008

(54) Ribosomal Rna Methyltransferases Rima:Target Validation and Processes for Deleloping an Inhibitor Assay and Identification of Candidate Inhibitors

(76) Inventors: Gaetano Thomas, Highland Park, NJ (US); Kalyan Das, Edison, NJ (US); Eddy Arnold, Belle Mead, NJ (US)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 10/536,705

(22) PCT Filed: Jun. 26, 2004
(86) PCT No.: PCT/US04/20244

§ 371 Date: Mar. 8, 2006

Related U.S. Application Data
(60) Provisional application No. 60/482,722, filed on Jun. 27, 2003.

(51) Int. Cl. C12N 9/10; C12Q 1/68

(52) U.S. Cl. 435/6; 435/193

(57) ABSTRACT

The invention provides a target and methods for specific binding and inhibition of RlmA proteins from bacterial specie The invention is directed to a method for identifying compounds that bind to a bacterial RlmA-binding pocket, comprising preparing a reaction solution comprising the compound to tested and an entity comprising a bacterial RlmA-binding pocket and detecting presence or amount of binding. The invention has applications in control of bacterial gene expression, control bacterial growth, antibacterial chemistry, and antibacterial therapy.

### CROSS-REFERENCE TO RELATED APPLICATIONS

This application claims priority to provisional application: 06/482,722 filed Jun. 27, 2003, the contents of which are incorporated herein by reference.

### GOVERNMENT SUPPORT

This invention was supported with U.S. Government funds (NIH P50-GM62413). Therefore, the Government may have rights in the invention.

### BACKGROUND ART

Bacterial infections remain among the most common and deadly causes of human disease. Infectious diseases are the third leading cause of death in the United States and the leading cause of death worldwide (Binder et al., Science 284:1311-1313 (1999)). The emergence of antibiotic-resistant bacterial strains is a primary clinical concern worldwide. It has been shown that bacterial pathogens can acquire resistance to first-line and even second-line antibiotics. (See, Stuart B. Levy, The Challenge of Antibiotic Resistance, in Scientific American, 46-53 (March, 1998); Walsh, C. (2000) Nature 406, 775-781; Schluger, N. (2000) Int. J. Tuberculosis Lung Disease 4, S71-S75; Raviglione et al., (2001) Ann. NY Acad. Sci. 953, 88-97). New approaches to drug development are necessary to combat the ever-increasing number of antibiotic-resistant pathogens.

The present invention provides one such approach, which involves a method of screening for compounds useful for the development of antibacterial drugs that work in combination with macrolide antibiotics that bind to the large ribosomal subunit, for which resistance is developed due to methylation of rRNA by RlmA^{I} or RlmA^{II}.

Certain modifications of bacterial ribosomal RNA (rRNA) are known to improve translational efficiency of the ribosome as well as confer resistance to ribosome-targeting antibiotics. Most of the modifications, though occurring at different bases of rRNA, cluster around the catalytic center of the ribosome (Brimacombe et al. 7(1) Faseb J. 161 (1993); Ban et al. 289(5481) Science 905 (2000)). The macrolide antibiotics (e.g., streptomycin, erythromycin, tylosin, spiramycin, etc.) bind to rRNA located in the large ribosomal subunit. Crystal structures of the large ribosomal subunit with bound macrolide antibiotics (Hansen et al. 10(1) Mol. Cell. 117 (2002)) have identified interactions between these antibiotic molecules and the RNA. The nucleotide bases G745, G748, and G2058 are clustered at the peptide exit channel. Modifications of these bases confer resistance to macrolide antibiotics as well as enhance ribosomal activity. A2058 is either monmethylated by the enzyme ErmN (TirD) or dimethylated by ErmE (Liu and Douthwaite 46(6) Antimicrob. Agents Chemother. 1629 (2002). Co-appearance of methylated G748 or (G745) with methylated A2058 (Weisbium 39(3) Antimicrob. Agents Chemother. 577 (1995); Liu and Douthwaite (2002)) confers high-level resistance to a wide range of certain macrolide drugs.

The bacterial RlmA (rRNA large subunit methyltransferases) class of enzymes (RlmA^{I} and RlmA^{II}) catalyzes N1-methylation of a guanine base (G745 in Gram-negative and G748 in Gram-positive bacteria) of hairpin 35 of 23S rRNA. Applicants' invention provides that the inhibition of methylation of G745 or G748 by RlmA would prevent the appearance of resistance to a number of macrolide antibiotics. Therefore when proposed RlmA inhibitor is given in combination with such a macrolide antibiotic, the bacteria will fail to develop resistance by the rRNA methylation and the macrolide antibiotic will continue to be effective.

### SUMMARY OF THE INVENTION

The present invention exploits Applicants' discoveries regarding the three-dimensional structure of Eschericia coli RlmA and particularly a deep, well-characterized binding pocket formed by its heterodimeric structure which recognizes and binds to a specific region of the bacterial rRNA. Applicants have chosen RlmA as a target for drug development in part because bacterial antibiotic resistance is developed due to methylation of rRNA by RlmA^{I} or RlmA^{II}. Applicants have discovered that the deep well-defined S-adenosyl-L-methionine—and rRNA-binding pocket of RlmA is a target for drugs which will interfere both with the process of binding rRNA and with the catalytic mechanism of methylation for the entire family of bacterial RlmA enzymes. Throughout the following specification, this region is referred to as the “target.” Applicants have determined the crystal structure of Escherichia coli RlmA^{I} at 2.8 Å resolution, providing the first three-dimensional structural information for the RlmA class of RNA methyltransferases. The dimeric protein structure exhibits features that provide new insights into its mechanism of action and molecular function. Each RlmA^{I} molecule has a Zn-binding domain, which is responsible for specific recognition and binding of its rRNA substrate, and a methyltransferase domain. The asymmetric RlmA^{I} dimer observed in the crystal structure has a well-defined deep W-shaped RNA-binding cleft. Two S-adenosyl-L-methionine (SAM) substrate molecules are located at the two valleys of the W-shaped RNA-binding cleft. The unique shape of the RNA-binding cleft, different from that of any other known RNA-binding protein, is highly specific and structurally complements the three-dimensional structure of hairpin 35 of bacterial 23S rRNA. Apart from the hairpin 35, parts of hairpins 33 and 34 also interact with the RlmA^{I} dimer. In addition, the unique 3D structure of the Zn-binding domain that interacts with rRNA makes this surface a good candidate for the design of RlmA-specific small molecule drugs.

Applicants' use of the term “RlmA” in the present invention is meant to refer to any and all bacterial RlmA^{I} and RlmA^{II} proteins, including, but not limited to those sequences shown in FIG. 1, and Applicants' use of the term “rRNA” in the present invention is meant to refer to any fragments of ribosomal RNA, including rRNArp35, a 16-base rRNA fragment corresponding to hairpin 35 that has been shown to bind to RlmA proteins (Lebars, I. et al., EMBO J. 2003 22 183-192), RNA-hairpins, and RNA-knots.

Because of the role of N1 methylation of G745 and G748 in developing resistance to macrolide antibiotics and in increasing translational efficiency of bacteria, Applicants propose RlmAs as novel targets for developing antibiotics, particularly as co-drugs of macrolide antibiotics. Applicants further have described a model of the RlmA-rRNA complex, which allows identification of specific protein-RNA interactions which are drug targets, and surface features within the deep cleft that can be used for drug design and/or the design of compound libraries for high-throughput screening.

Accordingly, one aspect of the present invention is directed to a method for identifying compounds that bind to a bacterial RlmA binding pocket domain, comprising preparing a reaction system comprising the compound to be tested and an entity containing a bacterial RlmA protein or a binding pocket domain thereof, and an rRNA that binds said protein or binding domain; and detecting the extent of binding between the RlmA protein and the rRNA, wherein reduced binding between the RlmA protein and the rRNA in the presence of the compound relative to a control is indicative of inhibitory activity of the compound against a bacterial strain. These compounds are then tested for their ability to enhance the inhibitory activities of various macrolide antibiotics (or other antibiotics) against specific bacterial strains, and to suppress macrolide (or other) antibiotic resistance. The compounds identified as having antibiotic-enhancing and/or antibiotic-resistance-suppressing activities against a bacterial strain can be further tested to determine whether they would be suitable as drugs. In this way, the most effective inhibitors of bacterial replication and/or co-drugs to be used in combination with macrolide (or possibly other) antibiotics can be identified for use in subsequent animal experiments, as well as for treatment (prophylactic or otherwise) of bacterial infection in animals, including humans. Similar compositions containing macrolide antibiotics and the RlmA inhibitors as co-drugs could be used as agents for disinfection in a wide variety of applications such as sterilization, and disinfection of skin, surfaces, objects handled by many people, etc. to prevent the spread of antibiotic-resistant bacteria.

Accordingly, another aspect of the present invention is directed to a method of identifying compounds having inhibitory activity and/or antibiotic-enhancing activities against a bacterial strain, comprising:

a) preparing a reaction system comprising an RlmA protein of a bacterial strain or an rRNA binding domain thereof, a rRNA fragment (e.g. rRNAhp35) that binds said protein or binding domain thereof, and a candidate inhibitor compound;

b) detecting extent of binding between the RlmA protein and the rRNA, wherein reduced binding between the RlmA protein and the rRNA in the presence of the compound relative to a control is indicative of inhibitory activity of the compound against the bacterium; and

c) determining extent of a compound identified in b) as having inhibitory activity to inhibit growth of a bacterial strain in vitro and/or to enhance the antibiotic activities of, and/or suppress the development of resistance to, macrolide or other antibiotic drugs. In some embodiments, the method further entails d) determining extent of a compound identified in c) as inhibiting growth of a bacterial strain in vitro, either alone or in combination with other antibiotics, or to inhibit replication of a bacterial strain in a non-human animal.

A further aspect of the present invention is directed to a method of preparing a composition for inhibiting growth of a bacterial strain in vitro or in vivo, comprising:

a) preparing a reaction system comprising an RlmA protein of a bacterial strain or a rRNA binding domain thereof, an rRNA that binds said protein or binding domain thereof, and a candidate compound;

b) detecting extent of binding between the RlmA protein and the rRNA, wherein reduced binding between the RlmA protein and the rRNA in the presence of the compound relative to a control is indicative of inhibitory activity of the compound against the bacterial strain;

c) determining extent of a compound identified in b) as having inhibitory activity to inhibit growth of a bacterial strain in vitro, and/or to enhance the antibiotic activities of, and/or suppress the development of resistance to, macrolide or other antibiotic drugs;

d) determining extent of a compound identified in c) as inhibiting growth of a bacterial strain in vitro, to inhibit replication of a bacterial strain in a non-human animal and/or to enhance the antibiotic activities of, and/or suppress the development of resistance to, macrolide or other antibiotic drugs; and

e) preparing the composition by formulating a compound identified in d) as inhibiting replication of a bacterial strain in a non-human animal, in an inhibitory effective amount, with a carrier.

In each of the above aspects of the present invention, some embodiments entail labeling the RlmA protein or the rRNA with a fluorescent molecule, and then determining extent of binding via fluorescent resonance energy transfer or measurements of fluorescence polarization anisotropy, or other fluorescence measurements. In other embodiments, the control is extent of binding between the rRNA and the RlmA protein or a rRNA binding domain. Yet still other embodiments entail methods of using a bacterial RlmA protein:rRNA complex formation in screening for or optimizing inhibitors. These embodiments include, but are not limited to NMR chemical shift perturbation of the RlmA protein, X-ray structure determination of RlmA-inhibitor complexes, gel filtration chromatography, sedimentation equilibrium measurements, sedimentation velocity measurements, hydrogen-deuterium exchange measurements using NMR or mass spectrometry, static light scattering measurements, dynamic light scattering measurements, X-ray crystallographic studies for screening compounds for binding to the RlmA protein or an RlmA protein-rRNA complex (with or without bound S-adenosylmethionine), and virtual screening using the structure of the RlmA protein and the model of the RlmA protein-rRNA complex.

A further aspect of the present invention is directed to a composition comprising a reaction mixture comprising a complex of a bacterial RlmA protein of a bacterial strain, or an rRNA binding fragment thereof, and an rRNA that binds said protein. In some embodiments, the composition further contains a candidate or test compound being tested for inhibitory, antibiotic resistance suppressing, and/or antibiotic-enhancing activities against Gram-negative or Gram-positive bacteria.

A still further aspect of the present invention is directed to a method of identifying a compound that can be used to treat bacterial infections, either alone or in combination with other antibiotics, comprising using the structure of a bacterial RlmA protein or an rRNA binding fragment thereof, and the three dimensional coordinates of a model of the RlmA protein:rRNA complex in an experimental or virtual drug screening assay.

It is anticipated that compounds identified according to the target and method of this invention would have applications not only in antibacterial therapy, but also in: (a) identification of bacterial RlmA (diagnostics, environmental-monitoring, and sensors applications), (b) labeling of bacterial RlmA (diagnostics, environmental-monitoring, imaging, and sensors applications), (c) immobilization of bacterial RlmA (diagnostics, environmental-monitoring, and sensors applications), (d) purification of bacterial RlmA (biotechnology applications), (e) regulation of bacterial gene expression (biotechnology applications), and (f) antisepsis (antiseptics, disinfectants, and advanced-materials applications).

These and other aspects of the present invention will be better appreciated by reference to the following drawings and Detailed Description.

### BRIEF DESCRIPTION OF THE DRAWINGS

FIG. 1A illustrates the amino acid sequence alignment (Gouet, P., Courcelle, E., Stuart, D. I. & Metoz, F. (1999) Bioinformatics 15, 305-308) of selected RlmA^{I} enzymes from Gram-negative (top 6 sequences) and RlmA^{II} enzymes from Gram-positive (bottom 4 sequences) bacteria. Conserved amino acids are in boxes. The secondary structure elements of E. coli RlmA^{I} (RRMA_ECOLI) determined in this work are mapped onto the alignment. FIG. 1B illustrates a ribbon diagram (Carson, M. (1997) Ribbons (Academic Press, New York) of the E. coli RlmA^{I} monomer structure. The three-strand smaller anti-parallel β-sheet is a part of the Zn-binding domain and the larger eight-stranded mixed β-sheet is the backbone of the MTase domain. The helices, except helix α5, are bundled into two groups. Helices α1, α2, α3, and α4 are in Group 1 and helices α6, α7, α8, η1 are in Group 2. The helix η1 is the only 3₁₀-helix in the structure. The S-adenosylmethionine (SAM) substrate is located at the center of the MTase domain as observed from our X-ray crystallographic study.

FIG. 2A illustrates a ribbon representation of an asymmetric dimer, as found in the crystal structure, showing a well-defined RNA-binding cleft. The deep W-shaped cleft has two Zn-fingers at the top and two SAM molecules at the bottom. FIG. 2B illustrates a stereo view of the novel Zn-finger motif of RlmA^{I}. FIG. 2B illustrates a stereo view of SAM binding region of an RmlA^{I} molecule. The |Fo|−|Fc|electron density mesh covering the SAM molecule was calculated at 2.8 Å resolution based on the phasing by protein atoms only.

FIG. 3. Superposition of the EmrC′ rRNA MTase structure (Schluckebier, G., Zhong, P., Stewart, K. D., Kavanaugh, T. J. & Abad-Zapatero, C. (1999) J. Mol. Biol. 289, 277-291.) onto the RlmA^{I} structure. Despite the fact that both the enzymes have superimposable MTase domains, their putative RNA-recognition domains are non-superimposible (on the left of the figure for EmrC′ on the right of the figure for RlmA^{I}) and have different tertiary folds.

FIG. 4A Top and FIG. 4B side view illustrations of the electrostatic potential surface of an E. coli RlmA^{I} dimer plotted using GRASP (Nicholls, A., Sharp, K. A. & Honig, B. (1991) Proteins 11, 281-296). The cleft formed by an RlmA^{I} dimer is largely positively charged and proposed to bind the substrate, hairpin 35 of bacterial 23S rRNA.

FIG. 5A The e. coli RlmA^{I} methyltransferase identifies an RNA-binding cleft formed by asymmetric arrangement of two RlmA^{I} monomers. The RNA substrate of the RlmA^{I} enzyme, Hairpin 35 region of 23S rRNA, structurally complements the RNA-binding cleft. FIG. 5B illustrates a stereo view of a modeled complex of RlmA^{I}:E. coli 23S rRNA fragment (Mueller, F., Sommer, I., Baranov, P., Matadeen, R., Stoldt, M., Wohnert, J., Gorlach, M., van Heel, M. & Brimacombe, R. (2000) J. Mol. Biol. 298, 35-59) containing hairpins 33, 34, and 35. The three-dimensional structure of this rRNA fragment complements the shape of the RlmA^{I} cleft formed by the MTase and Zn-binding domains. Nucleotide G745, which is methylated by RlmA^{I}, is located near the SAM-binding pocket of Molecule 1. FIG. 5C illustrates a comparison of conformation of the 23S rRNA fragment containing hairpins 33, 34, and 35 in three different structures of ribosome. The arrows indicate the angles between the domains of the rRNA fragments. FIG. 5D illustrates a schematic representation of the W-shaped RNA-binding cleft of RlmA^{I}, showing a proposed binding mode of hairpin 35 of 23S bacterial rRNA. The distances indicated correspond to E. coli RlmA^{I}.

### BEST MODE OF CARRYING OUT THE INVENTION

The present invention provides methods of designing specific inhibitors of the entire family of bacterial RlmA enzymes involved in the process of N1-methylation of bacterial ribosomal RNA (rRNA) and the enhancement of bacterial ribosomal activity. The invention provides targets for specific binding and inhibition of and methods for identifying agents to bind specifically and inhibit RlmA from bacterial species. The invention has applications in control of bacterial growth, control of bacterial protein translation, antibacterial chemistry, and antibacterial therapy.

MLS (macrolide, lincosamide, streptogramin B) antibiotics such as erythromycin, tylosin, and spiramycin are used in treating bacterial infections in humans and in animals (Roberts, M. C. (2002) Mol. Biotechnol. 20, 261-283). MLS antibiotics bind to the large ribosomal subunit (Vazquez, D. (1966) Biochim. Biophys. Acta 114, 277-288) and inhibit translation, possibly by blocking the protein exit-channel of the ribosome (Ban, N., et al., (2000) Science 289, 905-920; Brodersen, D. E., et al., (2000) Cell 103, 1143-1154; Schlünzen, F., et al., (2001) Nature 413, 814-821; Hansen, J. L., et al., (2002) Mol. Cell. 10, 117-128). The effectiveness of MLS antibiotics is increasingly limited by the emergence of resistant bacterial strains (Roberts, M. C. (2002) Mol. Biotechnol. 20, 261-283). Certain modifications of bacterial ribosomal RNA (rRNA) are known to confer resistance to MLS antibiotics (Baltz, R. H. & Seno, E. T. (1988) Annu. Rev. Microbiol. 42, 547-574; Vester, B. & Douthwaite, S. (2001) Antimicrob. Agents Chemother. 45, 1-12). One of the most common forms of bacterial rRNA modification is nucleotide methylation (Krzyzosiak, W., et al., (1987) Biochemistry 26, 2353-2364); for example, 10 methylations of 16S rRNA and 14 methylations of 23S rRNA nucleotides are reported (Rozenski, J., et al., (1999) Nucleic Acids Res. 27, 196-197) for E. coli. Although most of these modifications on rRNA occur prior to the formation of the ribosomal complex (Hansen, L. H., et al., (2001) J. Mol. Biol. 310, 1001-11), they primarily cluster around the catalytic center of the ribosome (Brimacombe, R., et al., (1993) FASEB J. 7, 161-167). Methylated nucleotide G748 functions synergistically with a methylated A2058 nucleotide to confer resistance to certain MLS antibiotics (Weisblum, B. (1995) Antimicrob. Agents Chemother. 39, 577-585; Liu, M. & Douthwaite, S. (2002) Proc. Natl. Acad. Sci. U.S.A. 99, 14658-14663).

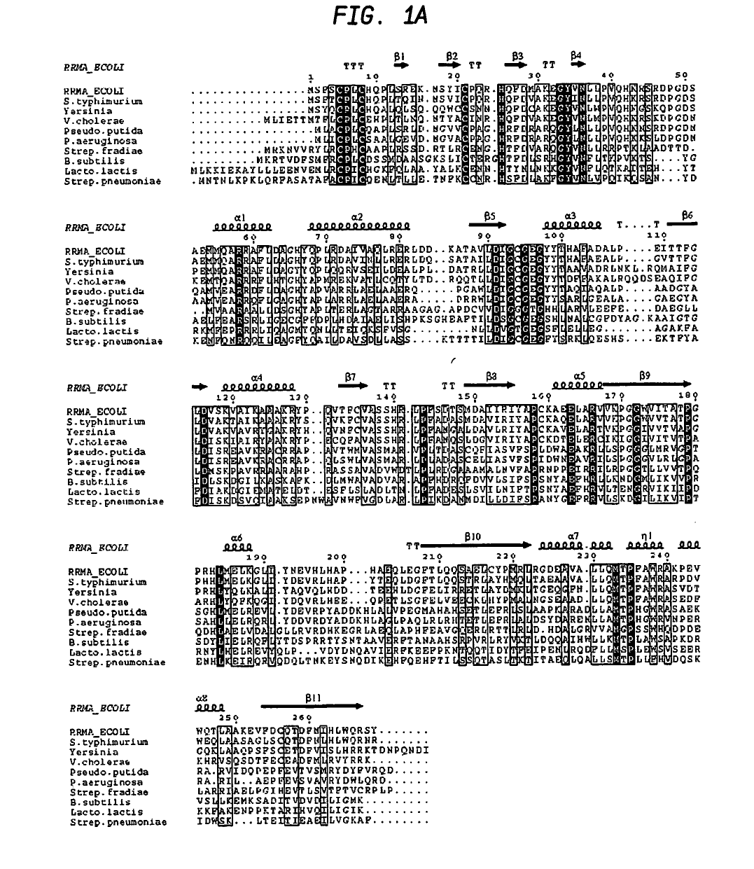

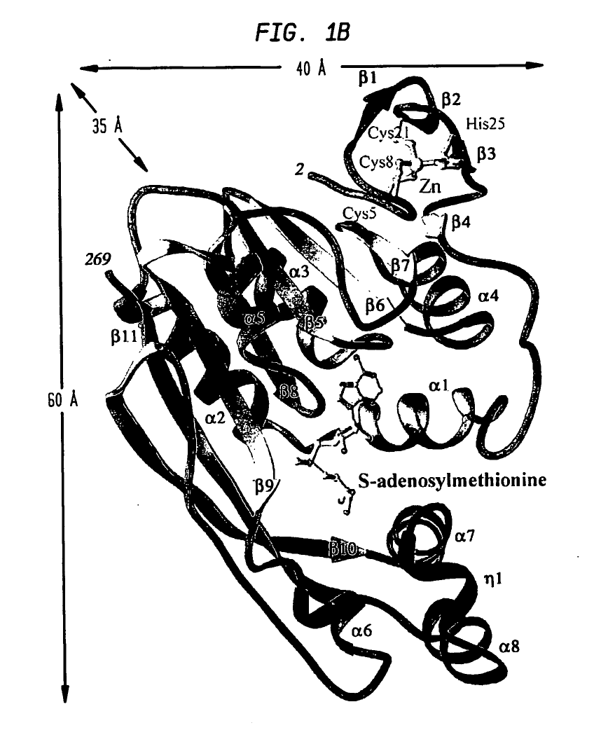

The N1-methylation of nucleotides G745 and G748 is carried out by rRNA large subunit methyltransferases RlmA^{I} and RlmA^{II} (formally known as rrma and TlrB) enzymes, respectively (Liu, M. & Douthwaite, S. (2002) Proc. Natl. Acad. Sci. U.S.A. 99, 14658-14663). RlmA enzymes are only present in bacteria (Fox, G. E., et al., (1980) Science 209, 457-463). However, the methyltransferase (MTase) domains of these enzymes exhibit amino acid sequence similarity with functionally related enzymes from eukaryotic and archea organisms and constitute a large structurally uncharacterized protein domain family. The RlmA class I (RlmA^{I}) enzyme is present in Gram-negative and the RlmA class II (RlmA^{II}) enzyme is present in Gram-positive bacteria (Gustafsson, C. & Persson, B. C. (1998) J. Bacteriol. 180, 359-365; Liu, M. & Douthwaite, S. (2002) Mol. Microbiol. 44, 195-204). Comparison of the amino acid sequences of RlmA^{I} and RlmA^{II} enzymes indicates that these enzyme classes are homologous (FIG. 1A); ˜29% of residues are conserved (Liu, M. & Douthwaite, S. (2002) Mol. Microbiol. 44, 195-204) across the species. Both the RlmA classes (I and II) contain a conserved MTase domain and use S-adenosyl-L-methionine (SAM) as the methyl group donor (Kagan, R. M. & Clarke, S. (1994) Arch. Biochem. Biophys. 310, 417-427). Despite functional similarity, RlmA enzymes from Gram-positive bacteria have a characteristic difference from those of Gram-negative bacteria: RlmA^{I} methylates G745 (Hansen, L. H., et al., (2001) J. Mol. Biol. 310, 1001-1110; Gustafsson, C. & Persson, B. C. (1998) J. Bacteriol. 180, 359-365), whereas RlmA^{II} methylates G748 (Liu, M., et al., (2000) Mol. Microbiol. 37, 811-820) at N1 position of the nucleotide bases. Both of these nucleotides, G745 and G748, are located in hairpin 35 of 23S rRNA.

E. coli RlmA^{I} is one of the best characterized RlmA enzymes (Gustafsson, C. & Persson, B. C. (1998) J. Bacteriol. 180, 359-365; Bjork, G. R. & Isaksson, L. A. (1970) J. Mol. Biol. 51, 83-100; Isaksson, L. A. (1973) Biochim. Biophys. Acta 312, 134-146; Isaksson, L. A. (1973) Biochim. Biophys. Acta 312, 122-133). Modifications to nucleotides of rRNA hairpins 33, 34, and 35 affect methylation by RlmA^{I} (Hansen, L. H., et al., (2001) J. Mol. Biol. 310, 1001-1110). A G745-deficient E. coli strain (Gustafsson, C. & Persson, B. C. (1998) J. Bacteriol. 180, 359-365) has a reduced growth rate as well as increased resistance to the ribosome-binding antibiotic viomycin, which inhibits by blocking translation of peptidyl-tRNA. Here Applicants report the X-ray crystal structure of E. coli RlmA^{I} at 2.8 Å resolution. In addition, Applicants describe modeling of the RlmA^{I}:rRNA complex aimed at understanding the specific recognition of this rRNA fragment, and the mechanism of N1-methylation of G745 and G748.

Applicants have discovered that RlmA^{I} from E. coli dimerized in a specific fashion to define a “W-shaped” binding cleft that would selectively recognize hairpin 35 of 23S RNA as its substrate. Two S-adenosylmethionine molecules, one per each RlmA^{I} monomer of a dimer, bind at the valleys (lower parts) of the W-shaped RNA-binding cleft at a distance of about 30 Å apart from each other. However, it appears that only one of the two RlmA^{I} molecules carries out the catalytic steps of methylation.

Applicants' invention predicts that the RNA-binding clefts of both RlmA^{I} and RlmA^{II} have similar folds and comparable shapes. High-specificity to hairpin 35 of 23S RNA and presence of a well defined deep RNA-binding cleft suggest that RlmAs could be targeted by substrate mimics. Dimerization of RlmAs appears to be crucial in defining the RNA-binding cleft. Active dimer formation may be prevented by small-molecule chemotherapeutic agents.

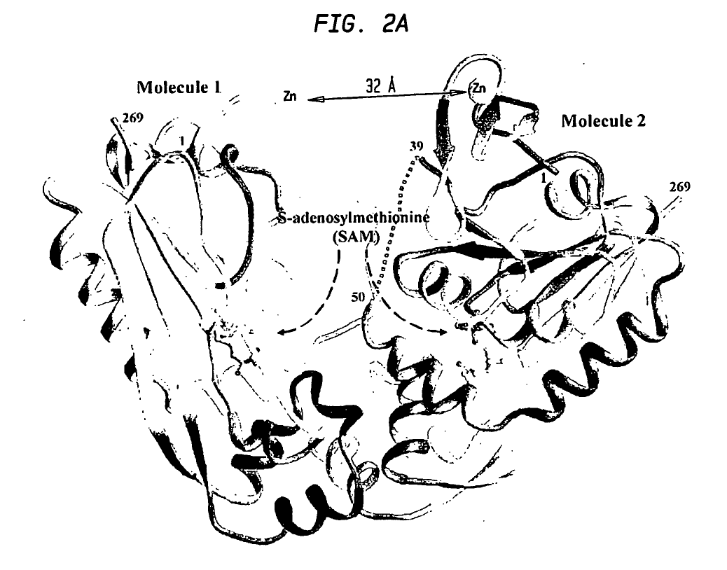

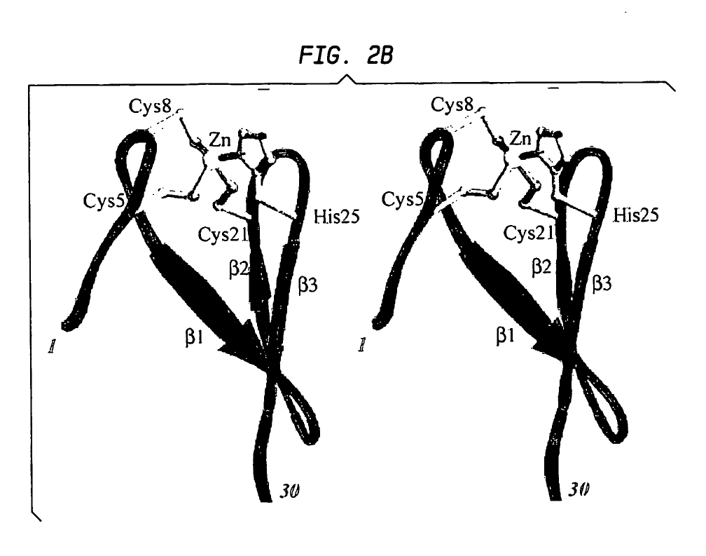

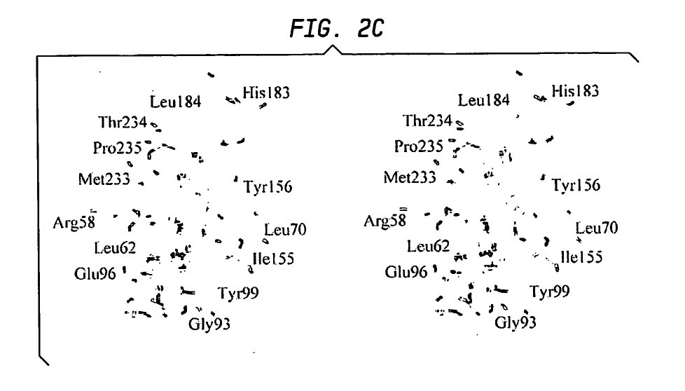

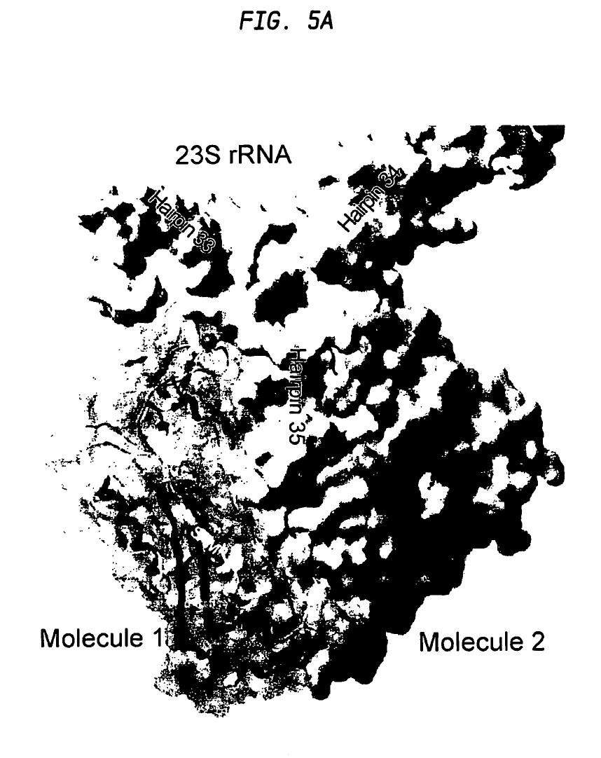

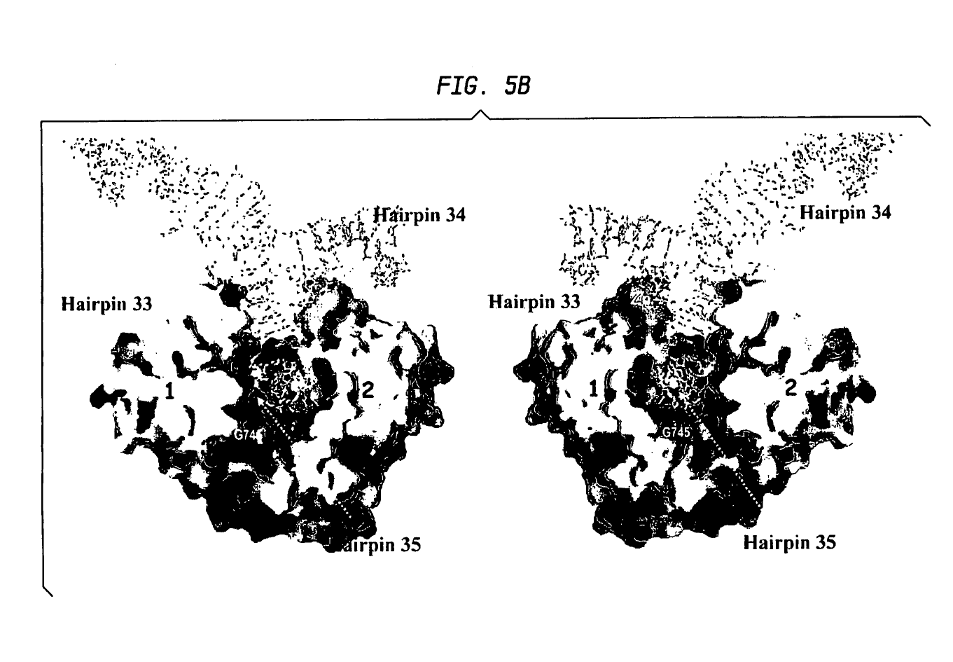

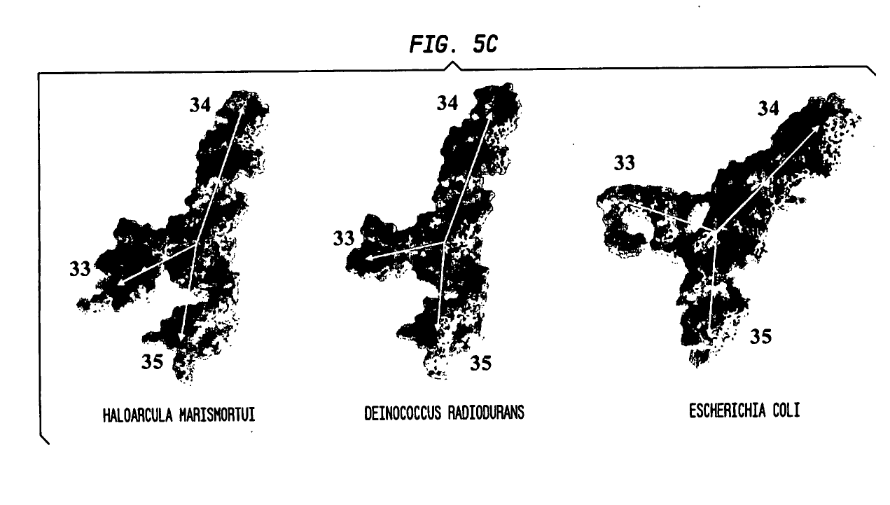

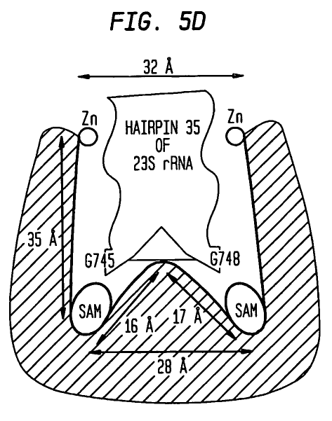

Crystal structure analysis and amino acid sequence comparisons of RlmAs show that the individual amino acids or structural motifs R(58)RAFL, Y(67), L(70), D(91)IGFCGEG, I(155)YAP, H(183)L, and L(230)LQMTP are responsible for binding of S-adenosylmethionine substrate. The Zn-binding domain (amino acid residues from 1 to 37), the linking loop between Zn-binding domain and methyltransferase domain (54-269), the structural motifs L(115)DVSK, and M(233)TP appear to recognize and interact with the RNA substrate. In the E. coli RlmA crystal structure, the linker region (amino acids 39-50) is partially ordered in one of the monomer and completely disordered in the other. The length and amino acid sequence of this region of RlmA^{I} is considerably different from that of RlmA^{II} enzymes suggesting that the region could be playing a role in orienting the RNA substrate in a specific way for RlmA^{I} which is different from that for RlmA^{II}. Applicants' invention provides that binding of RNA, binding of S-adenosylmethionine to RlmAs and dimerization of RlmAs are targets for inhibitor design. Applicants' invention also provides that inhibitors of RNA-binding and S-adenosylmethionine binding can be linked by a flexible linker group to design more effective inhibitors. The above listed conserved structural motifs, visualized by the three-dimensional structures illustrated in FIGS. 2 and 5, are targets for inhibitor binding.

Another aspect of Applicants invention provides that both RlmA^{I} and RlmA^{I} are favorable targets for antibacterial drug design. Applicants describe for the first time the three-dimensional structure of E. coli RlmA^{I} and identify a binding pocket formed by its heterodimeric structure which recognizes and binds to a specific region of rRNA. This binding pocket is a target for drugs which could interfere both with the process of recognition of the RNA substrate by the Zn-binding domains, binding rRNA into the W-shaped cleft and with the catalytic mechanism of methylation for the entire family of bacterial RlmA^{I} enzymes.

Another aspect of Applicants invention provides a model of the RlmA^{I}-rRNA complex, which allows identification of specific protein-RNA interactions which are potential drug targets. Applicants' invention also provides similar molecular target sites on a wide range of bacterial RlmA^{II} enzymes and its rRNA substrates.

### A Binding Assay and Process of Drug Discovery:

Applicants' invention provides methods for the discovery, design, and optimization of inhibitors targeting both RlmA^{I} and RlmA^{II}.

In one aspect of the present invention, compounds discovered or designed to prevent catalysis of N1-methylation by competing with RNA-binding and/or SAM-binding, and/or to interfere with active dimer formation are good candidates as lead compounds for antibacterial drug design. Such compounds are useful lead compounds for the development of antibacterial drugs that will work in combination with macrolide antibiotics (e.g., streptomycin, erythromycin, tylosin, spiramycin etc.), that bind to the large ribosomal subunit, against which resistance is developed due to methylation of rRNA by RlmA^{I} or RlmA^{II}.

In another aspect of the present invention, a high throughput in vitro assay (HTP-Assay) is developed to measure the affinity of binding various synthetic rRNA or RNA-knot substrates to RlmA^{I} and RlmA^{II} (the RlmA targets). These assays use standard methods of fluorescence resonance energy transfer (FRET), fluorescence polarization anisotropy with fluorophore-tagged RNA molecules or fluorophore-tagged RlmA target molecules to monitor interactions between these protein targets and various RNA molecules (RNA substrates), and to measure binding affinities. In fluorescence polarization anisotropy measurements, the signal-to-noise is determined by the change in the size of the fluorophore-tagged molecule upon complex formation. RlmA^{I} dimers are known to multimerize in solution to form large molecular weigh aggregates (Das et al. 2004. Proc. Natl. Acad. Sci. USA. 101:4041-4046). In a preferable embodiment, a relatively small rRNA molecule, like RNAhp35 fragment, which is known to bind RlmA proteins is fluorophore-tagged and combined with multimeric RlmA molecules, providing a large change in rotational correlation time upon complex formation and a corresponding high signal-to-noise ratio in fluorescence polarization anisotropy measurements.

In another aspect of the present invention, one or more HTP-Assays are used to screen compound libraries using conventional high-throughput screening technologies to identify molecules that will inhibit the interactions between the RlmA targets and the RNA substrates and between the enzyme monomers in the formation of active dimer. These compound libraries are obtained through collaborations with one or more biotechnology companies with expertise in this area, or purchased from commercial sources.

This process uses both random compound libraries and biased compound libraries designed using the particular structural features of the known RlmA target-RNA substrate interaction sites that have been deduced from Applicants' structural data.

In another aspect of the present invention, binding sites on the surface of RlmA targets and the RNA substrates of small molecule inhibitors identified by HTP screening are characterized using chemical shift perturbation NMR experiments. This could involve determination of a partial set of NMR resonance assignments for RlmA targets and/or the RNA substrates. In one embodiment of the invention, these assignments area facilitated by uniform or selective labeling of RlmA protein with NMR-active ¹³C and/or ¹⁵N nuclei. The relative locations of binding sites provide data for the design of linkers to link together multiple initial inhibitor leads that bind to separate binding sites and for optimizing lead design. NMR chemical shift perturbation measurements can also be used without resonance assignments to detect RlmA-rRNA interactions, and to screen for small molecules that prevent these interactions.

Structural characterization of how the novel inhibitors of the present invention bind to RlmA proteins can be accomplished by obtaining X-ray crystal structures of co-complexes. These X-ray crystallography methods can be used to screen for small molecules which bind in the active site and/or RNA-binding pocket of RlmA. Such structural analysis determines the specific binding modes and inhibitor-protein interactions. This information is used in a structure-based drug design approach to develop improved inhibitors.

In another aspect of the present invention, the structures of RlmA targets, RNA substrates, inhibitor molecules, and complexes between these are used for virtual (in silico) screening of compound libraries to identify additional lead compounds. These are further optimized using conventional SAR approaches and designs based on the predicted and experimental structures of the complexes.

The novel inhibitors of the interaction between RlmA targets and the RNA substrates are tested for their ability to inhibit bacterial growth, particularly of drug resistant strains, in culture both in the presence and in the absence of macrolide antibiotics. In still another aspect, Applicants' invention provides the most effective-inhibitors of bacterial replication for subsequent animal studies and eventual human clinical testing or for general antiseptic purposes (sterilization, disinfection, etc.).

Applicants' invention also provides crystals of RlmA^{I} for use in screening candidate inhibitors and potential drug targets. These crystals can be used together with standard X-ray crystallography methods for screening compound libraries to find molecules which bind in the cleft, and which are potential inhibitors of rRNA-binding and/or methylation function. These studies also provide information on the orientations and binding modes of these compounds, which can be used as a basis for structure-based drug design. Applicants' invention also provides strategies to identify small-molecule inhibitors from compound libraries. By way of example, three strategies are described as follows: (a) selection of molecules that bind to RlmA, or a fragment thereof, in an RlmA protein-rRNA-dependent fashion, (b) selection of molecules that inhibit interactions between RlmA and rRNA or inhibit interactions between RlmA and SAM, and (c) screening for molecules that inhibit translation in a RlmA protein-rRNA-dependent fashion, or that can act together with other antibiotics to enhance their antibiotic activities and/or suppress the development of antibiotic resistance. In each case, the invention provides the use of a wild-type bacterial RlmA, or fragment thereof, as the test protein for binding/inhibition, and a derivative of a bacterial RlmA protein, or a fragment thereof, having at least one of a substitution, an insertion, or a deletion within the RlmA as the control protein for rRNA-dependence of binding/inhibition.

The invention also provides for a method of identifying a compound for use as an inhibitor of bacterial RlmA protein comprising: analyzing a compound or a compound library, that involves docking to, modeling of, geometric calculations with, and/or energetic calculations with, a portion of the structure of an RlmA protein from a bacterial species comprising at least one residue within the set of residues corresponding to, and alignable with, the target.

The invention provides for at least three drug-discovery assay methods: a) screening based on binding of a compound within the RlmA binding pocket of a bacterial RlmA or fragment thereof; b) screening based on inhibition of an activity associated with the RlmA binding pocket of a bacterial RlmA or fragment thereof; c) screening based on displacement of a compound, containing a detectable group, from the RlmA-binding pocket of a bacterial RlmA or a fragment thereof.

The RlmA of a bacterial strain, or an rRNA binding domain thereof, and rRNA which interact and bind are sometimes referred to herein as “binding partners.” Any of a number of assay systems may be utilized to test compounds for their ability to interfere with the interaction of their binding partners. However, rapid high throughput assays for screening large numbers of compounds, including but not limited to ligands (natural or synthetic), peptides, or small organic molecules, are preferred. Compounds that are so identified to interfere with the interaction of the binding partners should be further evaluated for antibacterial activity in cell based assays, animal model systems and in patients as described herein. The basic principle of the assay systems used to identify compounds that interfere with the interaction between RlmA, or a rRNA binding domain thereof, and rRNA involves preparing a reaction mixture containing the RlmA, or a rRNA binding domain thereof, and rRNA under conditions and for a time sufficient to allow the two binding partners to interact and bind, thus forming a complex.

In order to test a compound for inhibitory activity, the reaction is conducted in the presence and absence of the test compound, i.e., the test compound may be initially included in the reaction mixture, or added at a time subsequent to the addition of RlmA, or an rRNA binding domain thereof, and rRNA; controls are incubated without the test compound or with a placebo. The formation of any complexes between the RlmA, or an rRNA binding domain thereof, and rRNA is then detected. The formation of a complex in the control reaction, but not in the reaction mixture containing the test compound indicates that the compound interferes with the interaction of the RlmA, or an rRNA binding domain thereof.

Another aspect of the present invention comprises a method of using the three dimensional coordinates of the model of the complex for designing compound libraries for screening.

Accordingly, the present invention provides methods of identifying a compound or drug that can be used to treat bacterial infections or to disinfect bacterial contamination, either alone or in combination with other antibiotics. One such embodiment comprises a method of identifying a compound for use as an inhibitor of the RlmA, or an rRNA binding domain thereof and a dataset comprising the three-dimensional coordinates obtained from the RlmA, or an rRNA binding domain thereof. Preferably, the selection is performed in conjunction with computer modeling.

In one embodiment the candidate compound is selected by performing rational drug design comprising the three-dimensional coordinates determined for the RlmA, or an rRNA binding domain thereof. As noted above, preferably the selection is performed in conjunction with computer modeling. The candidate compound is then contacted with and interferes with the binding of the RlmA, or an rRNA binding domain thereof, and rRNA, and the inhibition of binding is determined (e.g., measured). A potential compound is identified as a compound that inhibits binding of the RlmA, or an rRNA binding domain thereof, and rRNA when there is a decrease in binding. Alternatively, the candidate compound is brought into contact with and/or added to a bacterial infected cell culture comprising a macrolide antibiotic, wherein the growth of the bacteria is determined. This aspect of Applicants' invention, provides a method wherein the inhibitors of RlmA would function as “co-drugs” together with macrolide antibiotics to make them more effective. Such macrolide antibiotics include for example, streptomycin, erythromycin, tylosin, spiramycin, etc. A candidate compound is identified as a compound that inhibits bacterial growth in the presence of a macrolide antibiotic when there is a decrease in the growth of the bacterial culture.

In a preferred embodiment, the method further comprises molecular replacement analysis and design of a second-generation candidate drug, which is selected by performing rational drug design with the three-dimensional coordinates determined for the drug. Preferably the selection is performed in conjunction with computer modeling. The candidate drug can then be tested in a large number of drug screening assays using standard biochemical methodology exemplified herein. In these embodiments of the invention the three-dimensional coordinates of the RlmA and the model of the RlmA:rRNA complex provide methods for (a) designing an inhibitor library for screening, (b) rational optimization of lead compounds, and (c) virtual screening of potential inhibitors.

Still another aspect of the present invention comprises a method of virtual screening for a compound that can be used to treat bacterial infections comprising using the structure of the RlmA, or an rRNA binding domain thereof, and rRNA.

### Isolation of RlmA:

The bacterial RlmA protein, or RlmA derivative, can be isolated from bacteria, produced by recombinant methods, or produced through in vitro protein synthesis. Thus, the present invention does not require that the RlmA proteins be naturally occurring. Analogs of the RlmA protein that are functionally equivalent in terms of possessing the rRNA binding specificity of the naturally occurring protein, may also be used. Representative analogs include fragments of the protein, e.g., the rRNA binding domain. Other than fragments of the RlmA protein, analogs may differ from the naturally occurring protein in terms of one or more amino acid substitutions, deletions or additions. For example, functionally equivalent amino acid residues may be substituted for residues within the sequence resulting in a change of sequence. Such substitutes may be selected from other members of the class to which the amino acid belongs; e.g., the nonpolar (hydrophobic) amino acids include alanine, leucine, isoleucine, valine, proline, phenylalanine, tryptophan, and methionine; the polar neutral amino acids include glycine, serine, threonine, cysteine, tyrosine, asparagine, and glutamine; the positively charged (basic) amino acids include arginine, lysine, and histidine; the negatively charged (acidic) amino acids include aspartic and glutamic acid.

Various compounds can be introduced to determine whether a tested compound binds to, inhibits an activity of, or displaces a detectable-group containing molecule from, the bacterial RlmA or RlmA derivative in a RlmA-binding pocket-dependent manner.

Tested compounds can include RNA or DNA fragments, peptides, natural products, and various chemical compounds. Additionally, with the known amino acid sequence for a particular RlmA, one of skill in the art could design specific inhibitors.

The assay can be performed in vivo or in vitro and thus does not necessarily require isolation of the RlmA.

The tested compounds can be chosen from chemical libraries, or a computer model can be used to choose compounds that are likely to be effective based on the known structure of the RlmA binding pocket of a bacterial RlmA and the structure of the compound.

The compounds can also be tested for competitive inhibition. Preferred strategies for identifying inhibitors include: 1) through affinity selection of phage-displayed linear and cyclic decapeptide libraries, and 2) through iterative deconvolution of solution-phase linear and cyclic D-hexapeptide libraries, (3) interative deconvolution of solution-phase linear RNA libraries, and (4) SELEX, a method for generating and selecting for RNA molecules which bind to RlmA. Wild-type Escherichia coli RlmA is the preferred test protein for binding and inhibition. One of a derivative of Escherichia coli RlmA having at least one substitution in the target is the preferred control protein. Deconvolution essentially entails the resynthesis of that combinatorial pool or mixture that are found to be active in screening against a target of interest. Resynthesis may result in the generation of a set of smaller pools or mixtures, or a set of individual compounds. Rescreening and iterative deconvolution are performed until the individual compounds that are responsible for the activity observed in the screens of the parent mixtures are isolated.

### X-Ray Crystallography Approach:

X-ray crystallography will be routinely used in finding the lead RlmA inhibitors. (1) Attempts will be made to co-crystallized RlmA enzymes with small RNA fragments, RNA mimics and RNA-binding inhibitors. (2) Small molecules and RNA mimics will be randomly screened by molecular modeling and the compounds that will show favorable in silico binding to the described unique binding pocket will be attempted to be co-crystallized with RlmA enzymes. (3) The RlmA enzymes will be either co-crystallized or soaked with different organic molecules (such as benzene) and the crystal structures will be determined (Mattos C and Ringe D. Nat. Biotechnol. 1996 595-599). The positions of the bound organic molecules will be used to design lead inhibitor. (4) Once RlmA inhibitors or antibodies will be found based on described biochemical assays, the molecules will be co-crystallized with RlmA proteins and their binding modes will be accessed from the crystal structures of the complexes. The structural information will be used to improve the binding affinity of the molecules to RlmA enzymes.(5) X-ray Crystallography will be used routinely in optimizing lead inhibitors.

### Phage-Display Approach:

Tens of millions of short peptides can be easily surveyed for tight binding to an antibody, receptor or other binding protein using an “epitope library” (See (1990) Science 249:386; (1990) Science 249:404; and (1990) Proc. Natl. Acad. Sci. 87:6378). The library is a vast mixture of filamentous phage clones, each displaying one peptide sequence on the virion surface. The survey is accomplished by using the RlmA protein to affinity-purify phage that display tight-binding peptides and propagating the purified phage in Escherichia coli. The amino acid sequences of the peptides displayed on the phage are then determined by sequencing the corresponding coding region in the bacterial DNA's.

“Fusion phage” are filamentous bacteriophage vectors in which foreign antigenic determinants are cloned into phage gene III and displayed as part of the gene III protein (pIII) at one tip of the virion. Fusion phage displaying short cloned peptides are infectious analogs of chemically synthesized mimotopes, with the key advantages of replicability and clonability. A large library of such phage—an “epitope library”—may display tens of millions of peptide epitopes. The idea of using fusion phage to develop an “epitope library” (Parmley and G. P. Smith, (1988) Gene 73:305) was inspired by the synthetic “mimotope” strategy of Gheysen et al. (See Synthetic Peptides as Antigens; Ciba Foundation Symposium 119, R. Porter and J. Wheelan, Eds. (Wiley, New York. 1986), pp. 131-149). The peptides can in effect be individually surveyed for binding to a binding protein like RlmA by affinity purifying reactive phage from the library, propagating individual phage clones, and sequencing the relevant part of their DNAs to determine the amino acid sequences of their displayed peptides. The epitope library represents a powerful approach to the study of the specificity of antibodies and other binding proteins. (See Scott and Smith (1990) Science 249:386; Devlin et al., (1990) Science 249:404; Ciwirla et al., (1990) Proc. Nat'l Acad. Sci. 87:6378; McLafferty et al., (1993) Gene 128:29; Alessandra et al., (1993) Gene 128:51; McConnell et al., (1994) Gene 151:115, which are incorporated herein by reference).

### Iterative-Deconvolution and Positional-Scanning Approaches:

See the following reference for a general discussion of iterative deconvolution: (Ostresh et al., (1996) Meths. Enzym. 267:220, which is incorporated herein by reference). The practical development of synthetic combinatorial libraries (SCLs) made up of tens of millions of compounds has proven to be a powerful source for the identification of novel biologically active compounds such as analgesics, antibacterials, antifungals, and enzyme inhibitors. (See Pinilla et al., (1994) Drug Dev. Res. 33:133; Pinilla et al., (1995) Pept. Sci. 37:221; Gallop et al., (1994) J. Med. Chem. 37:1233; Blondelle et al., (1995) J. Appl. Bacteriol. 78:39; Blondelle et al., (1994) Antimicrob. Agent Chemother. 38:2280; Ostresh et al., (1994) Proc. Nat'l. Acad. Sci. U.S.A. 91:11138; Houghten et al., (1992) Bio Techniques 13:412; Houghten et al., (1991) Nature 354:84).

Two approaches can be employed for the structural deconvolution of active compounds from assay data using the “iterative” approach and the “positional scanning” approach. In addition, two synthetic methods can be used for the incorporation of multiple functionalities at diverse positions, as first illustrated for peptides, (See Houghten et al., (1992) Bio Techniques 13:412; and Houghten et al., (1991) Nature 354: 84, which are incorporated herein by reference). The first synthetic method, known as the “divide, couple, and recombine” (DCR) (Id.) or “split resin” (Lam et al., (1991) Nature 354:82) method, has typically been used with the iterative deconvolution approach. The second synthetic method, which involves the use of a predefined chemical ratio of protected amino acids at each coupling step for incorporation of mixture positions, Ostresh et al., (1994) Biopolymers 34:1681) has been developed for use with the positional scanning deconvolution process (Pinilla et al., (1992) BioTechniques 13:901). This latter method offers the advantage that both defined and mixture positions are easily incorporated at any position in a sequence.

These synthesis and deconvolution methods have been used to identify individual active compounds in a wide variety of assays. (Pinilla et al., (1994) Drug Dev. Res. 33:133; Pinilla et al., (1995) Pept. Sci. 37:221).

Peptide libraries for iterative and positional-scanning approaches are prepared using the DCR process (Houghten et al., (1991) Nature 354:84) in conjunction with simultaneous multiple peptide synthesis (SMPS) (Houghten, (1985) Proc. Natl. Acad. Sci. U.S.A. 82:5131) also known as the “tea bag” approach. Standard t-butyloxycarbonyl (Boc)-based peptide synthesis protocols are typically used to couple protected amino acids (Bachem, Torrance, Calif.) to methylbenzhydrylamine (MBHA)-derivatized polystyrene resin (Peninsula, Belmont, Calif.). Fluorenylmethyloxycarbonyl (Fmoc)-based chemistry strategies can also be used.

### RNA Oligonucleotide Libraries:

Libraries of RNA oligonucleotides for use in screening can also be generated by combinatorial chemical synthesis. RNA molecules that bind to RlmA can be isolated form these libraries using affinity purification by RlmA, and then amplified and sequence by RT-PCR methods, in which the RNA sequence is first converted to DNA using reverse transciptase, the resulting DNA is then amplified by polymerase chain reaction (PCR) methods, and the amplified DNA is then cloned and sequenced by conventional DNA sequencing methods. The corresponding RNA molecules can then be chemically synthesized in sufficient quantities for assay, and evaluated for their ability to inhibit RlmA-rRNA interactions. Related SELEX methods can also be used in a similar way to identify RNA oligonucleotides that bind to RlmA and are potential inhibitors of RlmA-rRNA interactions. (Famulok, M.; Szostak, J. W., In Vitro Selection of Specific Ligand Binding Nucleic Acids. Angew. Chem. 1992, 104, 1001. (Angew. Chem. Int. Ed. Engl. 1992, 31, 979-988.); Famulok, M.; Szostak, J. W., Selection of Functional RNA and DNA Molecules from Randomized Sequences. Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX. Mol. Biol. Reports 1994, 20, 97-107; Burgstaller, P.; Famulok, M. Synthetic ribozymes and the first deoxyribozyme. Angew. Chem. 1995, 107, 1303-1306 (Angew. Chem. Int. Ed. Engl. 1995, 34, 1189-1192). See also http://www.lmb.uni-muenchen.de/groups/famulok/SELEX.html for a description of the SELEX method.

### Screening for an Inhibitor of Bacterial RlmA:

One aspect of the invention, as described above, provides HTP screening of molecules specific to the bacterial RlmA target. This can be done in many different ways well known in the art. For example, this could be done by attaching bacterial RlmA to the bottom of the wells of a 96 well plate at an appropriate concentration by incubating the RlmA in the well overnight at 4° C. Alternatively, the wells are first coated with compositions of polylysine that facilitates the binding of the bacterial RlmA to the wells. Following attachment, samples from a library of test compounds are added to the wells and incubated for a sufficient time and temperature to facilitate binding. Following the incubation, the wells are washed with an appropriate washing solution at 4° C. Increasing or decreasing salt and/or detergent concentrations in the wash varies the stringency of the washing steps. Detection of binding is accomplished using antibodies, biotinylation, biotin-streptavidin binding, and radioisotopes in RIA and/or ELISA assays. Binding to the bacterial RlmA target identifies a “lead compound”. Once a lead compound is identified, the screening process is repeated using compounds chemically related to the lead compound to identify compounds with the tightest binding affinities. Selected compounds having binding affinity are further tested in one of two assays These assays use test compounds from 1) phage-displayed linear and cyclic decapeptide libraries, 2) iterative deconvolution of solution-phase linear and cyclic D-hexapeptide libraries, 3) RNA oligonucleotide libraries 4) RNA oligonucleotide molecules identified by SELEX methods, 5) small molecule libraries obtained commercially or provided by collaborators. The methods for generating these libraries (1)-(5) are described above.

A phage library can be used to test compounds that could bind to the RlmA-binding pocket of bacterial RlmA. The phage library is constructed in the N-terminal region of the major coat protein pVIII, as previously described (Felici et al., 1991). Phage affinity purification is performed utilizing the biopanning technique, as previously described by Parmley and Smith (1988). After the round of biopanning, 104 phage out of the initial 10¹⁰ are eluted from a streptavidin-coated plate. The phage is screened directly with a plaque assay. Positive plaques are eluted from nitrocellulose, the phage are amplified and sequenced, and their reactivity is further confirmed by dot-blot analysis. The amino acid sequences are then deduced.

Biologically active compounds are selected from large populations of randomly generated sequences. Libraries are made up of six-residue peptide sequences with amidated carboxy-termini and either acetylated or non-acetylated amino-termini. In total, 64,000,000 peptides are represented. The peptides are attached to a resin or alternatively cleaved from the resin, extracted and lyophilized before use. Each nonsupport-bound peptide mixture is typically used at a concentration of 1 mg/ml. After the mixture of libraries is screened for binding to bacterial RlmA, the remaining mixture positions are defined through an iterative enhancement and selection process in order to identify the most active sequence.

A rapid alternative method for identifying active compounds is the positional scanning approach. In this approach, if one uses a library made up of peptides, for example, each of the individual sub-libraries (one for each position along the peptide) that make up the positional scanning library is composed of 20 different peptide mixtures. Each individual peptide mixture, contains 3,200,000 (20⁵) different compounds; each of the six positional sub-libraries contains 64,000,000 (20×20⁵) different compounds; and the complete library contains 384,000,000 (6×20×20⁵) different compounds. Alternatively, each of the six individual sub-libraries can be examined independently and moved forward in an interactive fashion. The positional scanning approach can also be used with a library made up of RNA molecules.

The assay components and various formats that may be utilized are described in the subsections below.

### Assay Components:

The bacterial RlmA, or RlmA fragment or derivative, containing the RlmA-binding pocket, and an inhibitory compound specific to the RlmA-binding pocket, and rRNA binding partners are used as components in the assay may be derived from natural sources (e.g., purified from bacterial RlmA using protein separation techniques well known in the art); produced by recombinant DNA technology using techniques known in the art (see, e.g., Sambrook et al., 1989, Molecular Cloning: A Laboratory Manual, Cold Spring Harbor Laboratories Press, Cold Spring Harbor, N.Y.); and/or chemically synthesized in whole or in part using techniques known in the art (see, e.g., Creighton, 1983, Proteins: Structures and Molecular Principles, W. H. Freeman & Co., N.Y., pp. 50-60). The composition of the synthetic peptides may be confirmed by amino acid analysis or sequencing; e.g., using the Edman degradation procedure (see, e.g., Creighton, 1983, supra at pp. 34-49).

One of the binding partners used in the assay system should be labeled, either directly or indirectly, to facilitate detection of a complex formed between the bacterial RlmA-binding pocket, rRNA and an inhibitory compound specific to the RlmA. Any of a variety of suitable labeling systems may be used including but not limited to radioisotopes such as ¹²⁵I; enzyme labeling systems that generate a detectable calorimetric signal or light when exposed to substrate; and fluorescent labels. Fluorescent labels are preferred.

Where recombinant DNA technology is used to produce the bacterial RlmA, RlmA fragment, or derivative containing the RlmA-binding pocket, it may be advantageous to engineer fusion proteins that can facilitate labeling, immobilization and/or detection. For example, the coding sequence of the bacterial RlmA-binding pocket can be fused to that of a heterologous protein that has enzyme activity or serves as an enzyme substrate in order to facilitate labeling and detection. The fusion constructs should be designed so that the heterologous component of the fusion product does not interfere with binding of the bacterial RlmA-binding pocket and an inhibitory compound specific to the RlmA-binding pocket.

Indirect labeling involves the use of a third protein, such as a labeled antibody, which specifically binds to the bacterial RlmA-binding pocket. Such antibodies include but are not limited to, polyclonal, monoclonal, chimeric, humanized, single chain, Fab fragments and fragments produced by an Fab expression library.

For the production of antibodies, various host animals may be immunized by injection with the bacterial RlmA protein or fragment thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterial strain parvum.

Monoclonal antibodies may be prepared by using any technique that provides for the production of antibody molecules by continuous cell lines in culture. These include, but are not limited to the hybridoma technique originally described by Kohler and Milstein, (Nature, 1975, 256:495-497), the human B-cell hybridoma technique (Kosbor et al., 1983, Immunology Today, 4:72, Cote et al., 1983, Proc. Natl. Acad. Sci., 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity. Alternatively, techniques described for the production of single chain antibodies (e.g., U.S. Pat. No. 4,946,778) can be adapted to produce single chain antibodies specific to the bacterial RlmA-binding pocket. Alternatively, techniques described for the production of humanized antibodies can be adapted for the production of antibodies specific to the bacterial monomeric form of RlmA.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

ASSAY FORMATS: The assay can be conducted in a heterogeneous or homogeneous format. A heterogeneous assay is an assay in which reaction results are monitored by separating and detecting at least one component during or following reaction. A homogeneous reaction is an assay in which reaction results are monitored without separation of components.

In either approach, the order of addition of reactants can be varied to obtain different information about the compounds being tested. For example, test compounds that interfere with the interaction between the binding partners, e.g., by competition, can be identified by conducting the reaction in the presence of the test substance—i.e., by adding the test substance to the reaction mixture prior to or simultaneously with the bacterial RlmA-binding pocket and an inhibitory compound specific to the RlmA-binding pocket. On the other hand, test compounds that disrupt preformed complexes, e.g., compounds with higher binding constants that displace one of the binding partners from the complex, can be tested by adding the test compound to the reaction mixture after a complex between the binding partners has been formed.

In one example of a heterogeneous assay system, one binding partner—e.g.,_either the bacterial RlmA-binding pocket or an inhibitory compound specific to the RlmA-binding pocket—is anchored onto a solid surface, and the other binding partner, which is not anchored, is labeled, either directly or indirectly. In practice, microtiter plates are conveniently utilized. The anchored species may be immobilized by non-covalent or covalent attachments. Alternatively, an immobilized antibody specific for the bacterial RlmA-binding pocket may be used to anchor the bacterial RlmA to the solid surface. The surfaces may be prepared in advance and stored.

In order to conduct the assay, the non-immobilized binding partner is added to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the binding partner was pre-labeled, the detection of label immobilized on the surface indicates that complexes were formed. Where the binding partner is not pre-labeled, an indirect label can be used to detect complexes anchored on the surface; e.g., using a labeled antibody specific for the binding partner (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for an epitope on the bacterial RlmA-binding pocket to anchor any complexes formed in solution. Again, depending upon the order of addition of reactants to the liquid phase, test compounds which inhibit complex formation or which disrupt preformed complexes can be identified.

In other embodiments of the invention, a homogeneous assay can be used. In this approach, a preformed complex of the bacterial RlmA-binding pocket and an inhibitory compound specific to the RlmA-binding pocket is prepared in which one of the binding partners is labeled, but the signal generated by the label is quenched due to complex formation (see, e.g., U.S. Pat. No. 4,109,496). The addition of a test substance that competes with and displaces one of the binding partners from the preformed complex will result in the generation of a signal above background. In this way, test substances, which disrupt the bacterial RlmA-binding pocket and an inhibitory compound specific to the RlmA-binding pocket rRNA interaction can be identified.

For example, in a particular embodiment the bacterial RlmA protein, or an rRNA binding domain thereof, can be prepared for immobilization using recombinant DNA techniques described supra. Its coding region can be fused to the glutathione-S-transferase (GST) gene using the fusion vector pGEX-5X-1, in such a manner that its binding activity is maintained in the resulting fusion protein. RlmA or an rRNA binding domain thereof can be purified and used to raise a monoclonal antibody, specific for RlmA or an RlmA fragment, using methods routinely practiced in the art and described above. This antibody can be labeled with the radioactive isotope ¹²⁵I, for example, by methods routinely practiced in the art. In a heterogeneous assay, e.g., the GST-RlmA fusion protein can be anchored to glutathione-agarose beads. rRNA can then be added in the presence or absence of the test compound in a manner that allows rRNA to interact with and bind to the RlmA portion of the fusion protein. After the test compound is added, unbound material can be washed away, and the RlmA-specific labeled monoclonal antibody can be added to the system and allowed to bind to the complexed binding partners. The interaction between RlmA and rRNA can be detected by measuring the amount of radioactivity that remains associated with the glutathione-agarose beads. A successful inhibition of the interaction by the test compound will result in a decrease in measured radioactivity.

Alternatively, the GST-RlmA fusion protein and rRNA can be mixed together in liquid in the absence of the solid glutathione-agarose beads. The test compound can be added either during or after the binding partners are allowed to interact. This mixture can then be added to the glutathione-agarose beads and unbound material is washed away. Again the extent of inhibition of the binding partner interaction can be detected by measuring the radioactivity associated with the beads.

In accordance with the invention, a given compound found to inhibit one bacterial strain may be tested for general antibacterial activity against a wide range of different strains of bacteria. For example, and not by way of limitation, a compound which inhibits the interaction of E. coli RlmA with rRNA by binding to the RlmA binding site can be tested, according to the assays described infra, against different strains of bacteria.

To select potential lead compounds for drug development, the identified inhibitors of the interaction between RlmA targets and RNA substrates may be further tested for their ability to inhibit replication of bacterial strains, first in culture and then in animal model experiments. The lowest concentrations of each inhibitor that effectively inhibit bacterial growth will be determined using high and low multiplicities of infection.

One aspect of the invention provides fluorescence resonance energy transfer (FRET)-based homogeneous assays to provide probe-labeled derivatives of an inhibitory compound specific to the RlmA-binding pocket. (Förster, 1948; reviewed in Lilley and Wilson. 2000; Selvin, 2000; Mukhopadhyay et al., 2001; Mekler et al., 2002; Mukhopadhyay et al., 2004). FRET occurs in a system having a fluorescent probe serving as a donor and a second fluorescent probe serving as an acceptor, where the emission wavelength of the donor overlaps the excitation wavelength of the acceptor. In such a system, upon excitation of the donor with light of its excitation wavelength, energy can be transferred from the donor to the acceptor, resulting in excitation of the acceptor and omission at the acceptor's emission wavelength.

With commonly used fluorescent probes, FRET permits accurate determination of distances in the range of ˜20 to ˜100 Å. FRET permits accurate determination of distances up to more than one-half the diameter of a transcription complex (diameter ˜150 Å; Zhang et al. 1999; Cramer et al., 2001; Gnatt et al., 2001).

A preferred assay involves monitoring of FRET between: a) one of a fluorescent probe or a chromophore incorporated nearby by or into a bacterial RlmA-binding pocket, and b) one of a fluorescent probe or a chromophore incorporated into a rRNA, small molecule, or rRNA mimic that binds within the RlmA-binding pocket.

In accordance with the invention, a given compound found to inhibit one bacterial strain may be tested for general antibacterial activity against a wide range of different bacterial species. For example, and not by way of limitation, a compound that inhibits the interaction of Escherichia coli RlmA-binding pocket, can be tested, according to the assays described infra, against any bacterium.

### Animal Model Assays:

Any of the inhibitory compounds, which are identified in the foregoing assay systems, may be tested for antibacterial activity in any one of the various microbiological assays known to the skilled worker in the field of microbiology.

The most effective inhibitors of bacterial RlmA identified by the processes of the present invention can then be used for subsequent animal experiments. The ability of an inhibitor to prevent bacterial infection can be assayed in animal models that are natural hosts for bacteria. Such animals may include mammals such as pigs, dogs, ferrets, mice, monkeys, horses, and primates. As described in detail herein, such animal models can be used to determine the LD₅₀ and the LD₅₀ in animal subjects, and such data can be used to derive the therapeutic index for the inhibitor of the bacterial rRNA-binding pocket/inhibitory compound specific to the rRNA-binding pocket.

### Pharmaceutical Preparations and Methods of Administration:

The identified compounds that inhibit bacterial replication can be administered to a patient at therapeutically effective doses to treat bacterial infection. A therapeutically effective dose refers to that amount of the compound sufficient to result in amelioration of symptoms of bacterial infection.

Toxicity and therapeutic efficacy of such compounds can be determined by standard pharmaceutical procedures in cell cultures or experimental animals for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of infection in order to minimize damage to uninfected cells and reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal infection, or a half-maximal inhibition) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

As used herein a “small molecule” is a compound that has a molecular weight of less than 15 kDa.

As used herein a “small organic molecule” is an organic compound [or organic compound complexed with an inorganic compound (e.g., metal)] that has a molecular weight of less than 3 kDa.

As used herein the term “about” preferably means within 10 to 15%, preferably within 5 to 10%. For example, an amino acid sequence that contains about 60 amino acid residues preferably contains between 51 to 69 amino acid residues, more preferably 57 to 63 amino acid residues.

As used herein the term “target” minimally comprises amino acid residues of an RlmA-binding pocket target set of residues.

The present invention contemplates isolation of nucleic acids encoding the target. The present invention further provides for subsequent modification of the nucleic acid to generate a fragment or modification of the target that will crystallize.

### Protein-Structure-Based Design of Inhibitors of Bacterial RlmA:

Once the three-dimensional structure of a crystal comprising a bacterial RlmA target is determined, a potential modulator of the target can be examined through the use of computer modeling using a docking program such as GRAM, DOCK, or AUTODOCK (Dunbrack et al., Folding & Design, 2:27-42 (1997)), to identify potential modulators of the bacterial RlmA target. This procedure can include computer fitting of potential modulators to the bacterial RlmA target to ascertain how well the shape and the chemical structure of the potential modulator will bind to either the individual bound subunits or to the bacterial RlmA target (Bugg et al., Scientific American, Dec.:92-98 (1993); West et al., TIPS, 16:67-74 (1995)). Computer programs can also be employed to estimate the attraction, repulsion, and steric hindrance of the subunits with a modulator/inhibitor (e.g., the bacterial RlmA target and a potential stabilizer).

Initially, compounds known to bind to the target—for example, an inhibitory compound specific to the RlmA-binding pocket—can be systematically modified by computer modeling programs until one or more promising potential analogs are identified. In addition, systematic modification of selected analogs can then be systematically modified by computer modeling programs until one or more potential analogs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Alternatively, a potential modulator is obtained by initially screening a random peptide library produced by recombinant bacteriophage (Scott and Smith, Science, 249:386-390 (1990); Cwirla et al., Proc. Natl. Acad. Sci., 87:6378-6382 (1990); Devlin et al., Science, 249:404-406 (1990)) or a library of RNA molecules generated by combinatorial synthesis. A peptide or RNA oligonucleotide selected in this manner would then be systematically modified by computer modeling programs as described above, and then treated analogously to a structural analog as described below.

Once a potential modulator/inhibitor is identified, it can be either selected from a library of chemicals as are commercially available from most large chemical companies including Merck, Glaxo Welcome, Bristol Meyers Squib, Monsanto/Searle, Eli Lilly, Novartis and Pharmacia UpJohn, or alternatively the potential modulator may be synthesized de novo. As mentioned above, the de novo synthesis of one, or even a group of, specific compounds is reasonable in the art of drug design. The potential modulator can be placed into a standard binding assay with RlmA or an active fragment thereof such as the target, for example. The subunit fragments can be synthesized by either standard peptide synthesis described above, or generated through recombinant DNA technology or classical proteolysis. Alternatively, the corresponding full-length proteins may be used in these assays.

For example, the bacterial RlmA target can be attached to a solid support. Methods for placing the bacterial RlmA target on the solid support are well known in the art and include such things as linking biotin to the target and linking avidin to the solid support. The solid support can be washed to remove unreacted species. A solution of a labeled potential modulator (e.g., an inhibitor) can be contacted with the solid support. The solid support is washed again to remove the potential modulator not bound to the support. The amount of labeled potential modulator remaining with the solid support and thereby bound to the bacterial RlmA target can be determined. Alternatively, or in addition, the dissociation constant between the labeled potential modulator and the bacterial RlmA target, for example can be determined. Suitable labels for bacterial RlmA target or the potential modulator are exemplified herein. In a particular embodiment, isothermal calorimetry can be used to determine the stability of the bacterial RlmA target in the absence and presence of the potential modulator.

In another aspect of the present invention, a compound is assayed for its ability to bind to the bacterial RlmA target. A compound that binds to the bacterial RlmA target then can be selected. In a particular embodiment, the effect of a compound on the RlmA target is determined. The potential modulator then can be added to a bacterial culture to ascertain its effect on bacterial proliferation. A potential modulator that inhibits bacterial proliferation then can be selected.

The present invention provides for assays for analysis of antibacterial activity, such as for example include a Minimal Bacteriocidal Concentration (MBC) assay, concerning defining the target of an inhibitory compound specific to the RlmA-binding pocket. Such assays are conducted in the presence of a macrolide antibiotic.

When suitable potential modulators are identified, a crystal can be grown that comprises the bacterial RlmA, or a fragment thereof, a macrolide antibiotic, and the potential modulator. Preferably, the crystal effectively diffracts X-rays for the determination of the atomic coordinates of the protein-ligand complex to a resolution of better than 4.0 Å. The three-dimensional structure of the crystal is determined by molecular replacement. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR, CNS, (Crystallography and NMR System, a next level of XPLOR), and AMORE (J. Navaza, Acta Crystallographics ASO, 157-163 (1994)). Once the position and orientation are known, an electron density map can be calculated using the search model to provide X-ray phases. Thereafter, the electron density is inspected for structural differences and the search model is modified to conform to the new structure. Using this approach, it will be possible to solve the three-dimensional structure of different bacterial target having pre-ascertained amino acid sequences. Other computer programs that can be used to solve the structures of the bacterial RlmA from other organisms include: QUANTA, CHARMM; INSIGHT; SYBYL; MACROMODEL; and ICM.

A candidate drug can be selected by performing rational drug design with the three-dimensional structure determined for the crystal, preferably in conjunction with computer modeling discussed above. The candidate drug (e.g., a potential modulator of bacterial RlmA) can then be assayed as exemplified above, or in situ. A candidate drug can be identified as a drug, for example, if it inhibits bacterial proliferation.

A potential inhibitor (e.g., a candidate antibacterial agent) would be expected to interfere with bacterial growth. Therefore, an assay that can measure bacterial growth may be used to identify a candidate antibacterial agent.

Methods of testing a potential bacteriostatic or bacteriocidal compound (e.g., the candidate antibacterial agent) in isolated cultures and in animal models are well known in the art, and can include standard minimum-inhibitory-concentration (MIC) and minimum-bacteriocidal-concentration (MBC) assays. In a preferred embodiment, assays would test the ability of RlmA inhibitors to minimize resistance to macrolide or other antibiotics and/or enhance the effectiveness of macrolide or other antibiotics. In animal models, the potential modulators can be administered by a variety of ways including topically, orally, subcutaneously, or intraperitoneally depending on the proposed use. Generally, at least two groups of animals are used in the assay, with at least one group being a control group, which is administered the administration vehicle without the potential modulator.

For all of the assays described herein further refinements to the structure of the compound generally will be necessary and can be made by the successive iterations of any and/or all of the steps provided by the particular screening assay.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

### EXAMPLES

The invention provides a target and methods for specific binding and inhibition of large ribosomal subunit from bacterial species. The invention has applications in control of bacterial gene expression, control of bacterial growth, antibacterial chemistry, and antibacterial therapy.

### Example 1

### Cloning, Expression, and Purification

E. coli gene rrmA coding for RlmA^{I} was cloned into a pET21d (Novagen) derivative, generating plasmid pER19-21. E. coli BL21 (DE3) pMGK, a rare codon enhanced strain, was transformed with pER19-21. A single isolate was cultured in MJ (Jansson, M., et al., (1996) J. Biomol. NMR 7, 131-141) minimal media, containing selenomethionine instead of methionine, to produce Se-Met labeled RlmA^{I} protein (Doublie, S., et al., (1996) FEBS Lett. 384, 219-221). Initial growth was carried out at 37° C. until the OD₆₀₀ of the culture reached 1.0. The incubation temperature was then decreased to 17° C., and protein expression was induced by the addition of IPTG (isopropyl-b-D-thiogalactopyranoside) at a final concentration of 1 mM. Following overnight incubation at 17° C., the cells were harvested by centrifugation.

Selenomethionyl labeled RlmA^{I} was purified by standard methods. Cell pellets were resuspended in lysis buffer [50 mM NaH₂PO₄ (pH 8.0), 300 mM NaCl, 10 mM imidazole, 5 mM b-mercaptoethanol] and disrupted by sonication. The resulting lysate was clarified by centrifugation at 26,000×g for 45 min at 4° C. The supernatant was loaded onto a Ni-NTA column (Qiagen) and eluted in lysis buffer containing 250 mM imidazole. Fractions containing the partially purified RlmA^{I} were pooled and loaded onto a gel filtration column (Superdex 75, Amersham Biosciences), and eluted in Buffer A [10 mM Tris (pH 7.5), 5 mM DTT, 10 mM NaCl, 0.02% sodium azide]. The resulting purified RlmA^{I} protein was buffer exchanged and concentrated in 10 mM Tris (pH 7.5), 5 mM DTT to 10 mg/ml. Sample purity (>97%) and molecular weight (31.5 kDa) were verified by SDS-PAGE and MALDI-TOF mass spectrometry, respectively. The yield of purified protein was approximately 100 mg per 1 liter bacterial culture.

### Example 2

### Crystallization

A sample of RlmA^{I} at a concentration of ˜1.0 mg/mL in 10 mM Tris-HCl was used for dynamic light scattering measurements using a ProteinSolutions' DynaPro light scattering device. Radius of the sample based on 25 consecutive readings was 344 Å with a polydispersity of ˜43% (a standard value for most crystallizing proteins is less than 25%). The calculated average molecular weight of the large RlmA^{I} aggregates observed in these measurements (radius ˜334 Å) is ˜1.33×10⁴ kDa, whereas, the molecular weight of an RlmA^{I} monomer is 30.4 kDa.

Crystallization conditions for the RlmA^{I} protein were surveyed using hanging drop vapor diffusion techniques and the Hampton Crystal Screen I & II and PEG/ION screen kits. Initial trials with protein concentrations of ˜10 mg/mL did not give any positive indications of crystals, and most of the drops precipitated. Use of a lower concentration of protein (˜6 mg/mL) yielded fiber-like micro crystals using Hampton Crystal Screen II #15 (0.5 M ammonium sulfate, 1.0 M lithium sulfate, and 0.1 M sodium citrate pH 6.5). After numerous optimization attempts, the hanging drop setup with 4 mg/mL protein in 10 mM Tris-HCl pH 7.5, 5 mM SAM, and 5 mM DTT produced the best crystals when vapor diffused against the above crystallization solution. The crystals grew to optimum size of 0.1×0.1×0.05 mm³ in about four weeks at 22° C.

### Example 3

### Data Collection and Structure Determination

Se-Met E. coli RlmA^{I} crystals were mounted on cryo-loops, cryoprotected by dipping in solution containing 20% ethylene glycol, and flash cooled in liquid-N₂. MAD (Multiple Anomalous Diffraction) data was collected at X12C NSLS, Brookhaven National Laboratory (BNL) from one flash-cooled crystal. The data (Table 1) were processed to 3.2 Å resolution using Denzo/Scalepack (Otwinowski, Z. & Minor, W. (2001) in The International Union of Crystallography Vol. F. Crystallography of Biological Macromolecules, eds. Rossmann, M. G. & Arnold, E. (Kluwer Academic Publishers, Boston), pp. 226-235). Another crystal with comparable dimensions was used to collect higher resolution data at the F1 beam line of the Cornell High Energy Synchrotron Source (CHESS), and processed at 2.8 Å resolution. Thirteen Se sites were found using the Direct Methods implemented in SnB 1.0 (Howell, P. L., et al., (2000) Acta Crystallogr. D Biol. Crystallogr. 56, 604-617). The phases were calculated at 3.5 Å resolution, by Solve 2.03 using the Se sites, and extended to 3.2 Å resolution using NCS averaging and solvent correction methods implemented in Resolve (Terwilliger, T. C. & Berendzen, J. (1999) Acta. Crystallogr. D Biol. Crystallogr. 55, 849-861). The electron density calculated at 3.2 Å resolution was well defined, and most of the amino acid residues could be modeled manually. Later, 2.8 Å resolution data was used to refine the structure. Cycles of model building (using O (Jones, T. A., et al., (1991) Acta Crystallogr. A 47, 110-119)) and refinement (initially using Refmac V 5.1.24 (Murshudov, G. N., et al., (1999) Acta Crystallogr. D Biol. Crystallogr. 55, 247-255) implemented in CCP4 V4.2.1 and later using CNS 1.1 (Brunger, A. T., et al., (1998) Curr. Opin. Struct. Biol. 8, 606-611) augmented the experimental phases and allowed identification of the remaining amino acid positions. The final model was refined to R=0.248 and R_{free}=0.296 (Table 1).

The RlmA^{I} molecules in the crystal lattice were arranged with 1222 space group symmetry having unit cell parameters a=107, b=122 and c=144 Å and α=β=γ=90°.

TABLE 1
Crystallographic parameters, X-ray data and refinement
statistics for E. coli RlmA^{I}.

| | Se-λ1 | Se-λ2 | Se-λ3 | λ_{high resolution} |
|---|---|---|---|---|
| Data collection facility | BNL X12C | | | CHESS F1 |
| Wavelength (λ) in Å | 0.97889 | 0.97874 | 0.9500 | 0.9160 |
| Resolution range (in Å) | 50.0-3.2 | 50.0-3.2 | 50.0-3.2 | 50.0-2.8 |
| Number of reflections | 28,604 (117,100) | 28,528 (118,011) | 28,354 (102,647) | 21,876 (88,268) |
| (Number of observations) | | | | |
| Completeness | 95.6 | 95.7 | 94.8 | 93.0 |
| Average I/σ(I) | 4.7 | 3.7 | 4.2 | 11.0 |
| ¹R_{merge} on I | 0.175 | 0.206 | 0.183 | 0.106 |
| Sigma Cut-off | I < −1σ(I) | I < −1σ(I) | I < −1σ(I) | I < −0.5σ(I) |

| | | |
|---|---|---|
| Mean figure of merit | 0.40 (40.0-3.5 Å resolution) | |
| Unit cell constants (Space | a = 107.10, b = 122.36, and c = 142.68 Å | a = 107.19, b = 122.28, |
| group) | (I222) | c = 143.14 Å |

| Data set used in structure refinement | | λ_{high resolution} |
|---|---|---|
| Resolution range | | 20-2.8 Å |
| Total number of reflections (R_{free} set) | | 21,804 (1,138) |
| Completeness (R_{free} set) | | 93% (5%) |
| Cutoff criteria | | \|Fₒ\| ≧ 1.0 σ(Fₒ) |
| Number of atoms refined (non-protein atoms) | | 4,345 (165) |
| ²R_{cryst} | | 0.248 |
| R_{free} | | 0.296 |
| Rms deviation | Bond length | 0.012 Å |
| | Bond angle | 1.7° |

¹R_{merge} = ΣₕₖₗΣᵢ|I(hkl)ᵢ − <I(hkl)>|/ΣₕₖₗΣᵢ <I(hkl)ᵢ>
²R_{cryst} = Σₕₖₗ|Fₒ(hkl) − kF_{c}(hkl)|/Σₕₖₗ|Fₒ(hkl)|, where Fₒ and F_{c} are observed and calculated structure factors, respectively.

Applicants' invention provides for the cloning, expression, purification protocol, crystallization conditions, and intrinsic properties of the crystals, such as, unit cell parameters, space group including sub- and super-space group.

The structure of RlmA^{I} revealed (1) a new mode of RNA binding, and (2) a new type of Zn-finger that had not been observed prior to our current crystallographic work.

### Example 4

### Overall Structure of RlmA^{I}

The crystal structure of E. coli RlmA^{I} is shown in FIG. 1B. The structure was determined by SeMet-MAD method and refined to 2.8 Å resolution. RlmA^{I} crystallized as a dimer per asymmetric unit (FIG. 2A), with dimensions 85×60×35 Å³. The two monomers (each having molecular weight 30.4 kDa and 269 amino acid residues) within the dimer have an unusual asymmetric arrangement in which one monomer relates to the other by ˜160° rotation about a 2-fold non-crystallographic symmetry axis. dimer contains a wide “W-shaped” cleft, a putative binding site for the rRNA substrate. The root mean square (rms) deviation for superposition of Ca-atoms of the two monomers is 1.1 Å.

The secondary structure of an RlmA^{I} monomer includes eleven β-strands, eight β-helices, and one 3/10-turn helix (FIG. 1B). The first three N-terminal b-strands form a small anti-parallel b-sheet, a part of a Zn-binding domain (FIG. 2B), and the remaining eight strands form a large twisted mixed b-sheet that contains a characteristic MTase fold. An N-terminal Zn-binding domain (amino acids 1 to 35) and a C-terminal MTase domain (amino acids 51 to 269) are connected by a flexible linker of 12-15 amino acids. This linker is partially ordered in Molecule 1 and completely disordered in Molecule 2 of the crystallographic dimer. In the MTase domain, the two C-terminal b-strands b10 and b11 are curved and unusually long (˜50 Å in length), each containing 14-15 amino acids.

The base of the W-shaped RNA-binding cleft is formed by two methlytransferase domains, one per monomer. Two valleys of the W-shaped cleft contain two SAM molecules, one bound to each monomer (FIG. 2A). The helices a6, a7, a8, and h1 ( 3/10-helix) as well as parts of helices a1 from each monomer are clustered to form the RlmA^{I} dimer interface. In addition to these interactions between RlmA^{I} monomomers, there are extensive interactions between RlmA, dimeric units in the crystal structure. The large b10-strand of Molecule 1 interacts with the b10-strand of a crystallographic symmetry related Molecule 2 to form an extended 16-strand b-sheet. These two distinct sets of intermolecular interactions for RlmA^{I} molecules, (i) between the monomer units of the dimer and (ii) between these dimmers, as seen in the crystal structure, might also exist in solution and could be responsible for formation of the large aggregates (of average radius 344 Å) observed in dynamic light scattering measurements.

### Example 5

### Zn-Binding Domain

The N-terminal 35 amino acid residues of RlmA^{I} form a Zn-binding domain which appears to be important in rRNA recognition. Within the Zn-binding domain, conserved amino acids Cys5, Cys8, Cys21, and His25 coordinate with a single Zn-ion. The presence of Zn-ion was evident from the crystallographic study and was further confirmed by inductively coupled plasmon resonance spectroscopy. The Zn-binding domain, that is present in all members of both the RlmA enzyme classes (FIG. 1A), has a novel Cys₃H is Zn-finger fold (FIG. 2B); its amino acid consensus sequence (Cys-Pro-X-Cys-12/13X-Cys-3/4X-His) and three-dimensional structure are different than those of previously characterized Zn-finger structures.

The two Zn-ions, positioned at the two top edges of the W-shaped RNA-binding cleft, are about 32 Å apart; two highly conserved Cys-Pro-Leu-Cys loops (amino acids 5-8, a part of the Zn-finger) are about 24 Å apart. Based on rRNA docking (as discussed later), the Cys-Pro-Leu-Cys loops and His25 appear to be involved in recognition and binding of the rRNA substrate, hairpin 35 region of 23S rRNA. The Zn-binding domain of Molecule 2 is less ordered with an average B-factor of 82 Å², compared with an average B factor of 52 Å² for the corresponding domain in Molecule 1. The Zn-binding domains, particularly of Molecule 2, and the loops joining them with the MTase domains may adjust their positions upon interacting with the rRNA substrate and could consequently be stabilized by RNA:protein interactions.

### Example 6

### SAM Binding

RlmA enzymes use SAM as methyl group donor (19). As mentioned above, SAM molecules are bound to the MTase domains of both the RlmA^{I} monomers (FIG. 2A). Difference electron density maps clearly define the mode of binding of SAM in the RlmA^{I} enzyme structure (FIG. 2C). Relatively higher B-factors of the SAM molecules, compared to those of the surrounding protein atoms, indicate partial occupancy (or positional disorder) of these substrate molecules. The amino acid residues that take part in SAM binding, including Arg58-Leu62, Tyr67, Leu70, Gly93-Tyr99, Ile155-Tyr156, His183-Leu184, and Met233-Pro235 (FIG. 2C), are either identical or of similar types in homologous RlmA^{I} and in RlmA^{I} enzymes (FIG. 1A). Most of the conserved amino acids interacting with the SAM molecule, except those in a1 helices, are located on structurally flexible regions such as polypeptide loops and the tips of helices pointing towards the SAM-binding region. The two SAM molecules, although bound in similar regions of the monomers of RlmA^{I} dimer, differ in their precise orientations and specific interactions with protein atoms. Presumably, binding of the RNA substrate is necessary for a SAM molecule to bind to the RlmA^{I} enzyme in a proper orientation for MTase catalysis.

### Example 7

### Comparison with Other rRNA Methyltransferase Structures

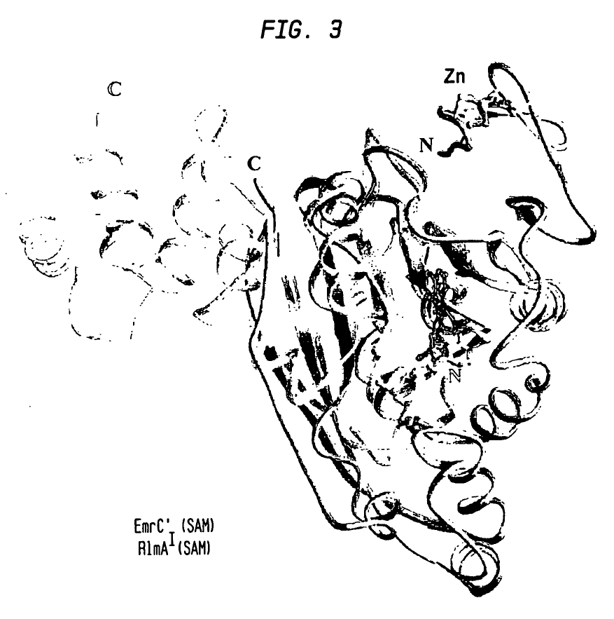

Crystal structures of bacterial rRNA MTases E. coli RlmB (Michel, G., et al., (2002) Structure (Camb.) 10, 1303-1315), Bacillus subtilis ErmC′ (Schluckebier, G., et al., (1999) J. Mol. Biol. 289, 277-291; Mosbacher, T. G., et al., (2003) J. Mol. Biol. 329, 147-157), and Streptomyces viridochromogenes AviRa (Mosbacher, T. G., et al., (2003) J. Mol. Biol. 329, 147-157) have been previously described. These enzymes are highly specific to their respective RNA substrates, parts of bacterial rRNA. Although the overall structures of these MTases are different, all three of these enzymes contain MTase domains that have a common Rossmann-type fold. The MTase domain of RlmA^{I} also has this characteristic fold. A Dali structural database search (Holm, L. & Sander, C. (1995) Trends Biochem. Sci. 20, 478-480) identifies the MTase domain of ErmC′ (Bussiere, D. E., et al., (1998) Biochemistry 37, 7103-7112) as one of the top structural analogs (Z=13.2; 140 Ca atoms superimposed with rms deviation of 1.8 Å) of RlmA^{I}. Despite the structural similarity of the SAM binding/MTase domains of RlmA^{I} and ErmC′ (FIG. 3), the sequence identity in the structurally-superimposed regions is only 9%. Due to a low sequence identity with know structures, the fold of the MTase domain of RlmA enzymes could not be recognized prior to this structure determination.

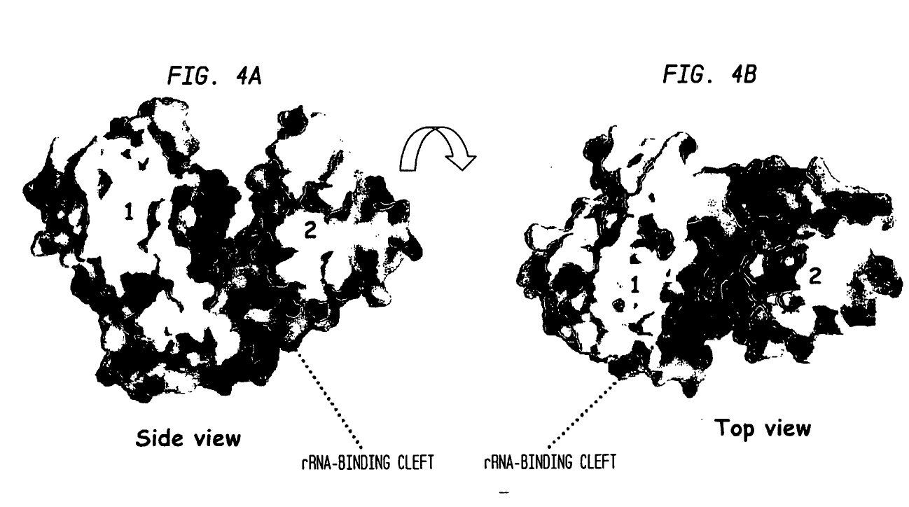

A comparison of the overall structures of RlmA^{I} and ErmC′ provides some valuable insights (FIG. 3). The relative positions and orientations of the bound SAM molecules in RlmA^{I} differ significantly from those of ErmC′ structure (Bussiere, D. E., et al., (1998) Biochemistry 37, 7103-7112). In addition, the putative rRNA-recognizing domains (e.g., the Zn-binding domain of RlmA^{I}) of the two enzymes have different tertiary fold and are positioned differently with respect to superimposed MTase domains (FIG. 3). This structure comparison suggests differences in the mode of rRNA-substrate recognition by the MTase enzymes, despite a plausible common catalytic mechanism. These structural differences provide a basis for these enzymes' specificities to their respective substrates, different parts of bacterial rRNA. Among the three rRNA MTase structures discussed above, the reported structures of EmrC′ (Bussiere, D. E., et al., (1998) Biochemistry 37, 7103-7112) and AviRa (Mosbacher, T. G., et al., (2003) J. Mol. Biol. 329, 147-157) have no well-defined RNA-binding cleft/pocket and the RNA-binding cleft that has been described for dimeric RlmB (Michel, G., et al., (2002) Structure (Camb.) 10, 1303-1315) is very different than that of RlmA^{I} (FIG. 4).

### Example 8

### Binding of rRNA Substrate

The W-shaped putative rRNA-binding cleft (FIG. 2A) is comprised of conserved amino acid residues from both monomers of an asymmetric RlmA^{I} dimer. Two Zn-fingers are at the top and the two SAM molecules are at the bottom of the cleft. At the bottom of the cleft, helices a1 from each monomer together form a ridge that separates the two SAM-binding pockets. The W-shaped cleft is lined with a positively charged electrostatic surface suitable for interactions with polyanionic nucleic acids (FIG. 4). The unusual asymmetric arrangement of RlmA^{I} molecules in its dimer appears to be functionally relevant in creating the specific shape of the rRNA-binding cleft. The shape of the cleft is unique and different from that of previously reported RNA-binding proteins.

Considering the clearly identifiable rRNA binding cleft of RlmA^{I}, efforts were made to model the structure of its complex with hairpin 35 of 23S rRNA. The relevant parts from structures of the large ribosomal subunit of E. coli (by cryo-EM at 7.5 Å; Mueller et al. (2000). J. Mol. Biol. 298, 35-59; PDB Id. 1C2W), of Haloarcula marismortui (by X-ray at 2.4 Å (Ban, N., et al., (2000) Science 289, 905-920); PDB Id. 1FFK), eubacterial strain Deinococcus radiodurans (by X-ray at 3.1 Å (Schlünzen, F., et al., (2001) Nature 413, 814-821); PDB Id. 1JZX), Thermus thermophilus (by X-ray at 5.5 Å (Yusupov, M. M., et al., (2001) Science 292, 883-896); PDB Id. 1G1X), and the structure of hairpin 35 from Streptococcus pneumonia rRNA (by NMR (Lebars, I., et al., (2003) EMBO J. 22, 183-92); PDB Id. 1MT4) were docked into the cleft of the RlmA^{I} dimer (FIG. 4). Manual docking of the portion of the E. coli rRNA structure containing hairpins 33, 34, and 35 (nucleotides 692 to 770) (Mueller, F., et al., (2000) J. Mol. Biol. 298, 35-59), into the RNA-binding cleft of RlmA^{I} provides a unique complementary match (FIG. 5A). In this modeled complex structure, hairpin 35 is completely buried in the cleft. The RNA-bulge (knot) linking the three hairpins (33, 34, and 35) sits over the Zn-finger regions of the cleft suggesting that the two Zn-fingers are responsible (i) for recognition of the rRNA substrate structure and (ii) for placing the hairpin 35 in the W-shaped cleft. This model of the rRNA:RlmA^{I} complex (FIG. 5A) is consistent with previously reported biochemical studies by showing that, in addition to the hairpin 35, nucleotides from the adjacent hairpins 33 and 34 interact with RlmA^{I}; most of the interacting nucleotides are from hairpin 35 and the RNA-bulge whereas, the top part of hairpin 34 is not interacting with the RlmA^{I} dimer. Interestingly, in this model of the protein:rRNA complex (FIG. 5A), the base of nucleotide G745 (the target for methylation in Gram-negative bacteria) is positioned in close proximity to the SAM-binding pocket of Molecule 1. The excellent unique fit of this rRNA fragment in the dimeric structure of RlmA^{I} suggests that the observed structural asymmetry of the dimer is indeed required for unique recognition and binding of the rRNA substrate.

As shown in FIG. 5B, the relative orientations of rRNA hairpins 33, 34, and 35 are somewhat different but related in different ribosome structures. An important difference in the arrangements of these hairpins is the angle between hairpins 33 and 35. This angle appears to be critical for binding of the rRNA fragment to the RlmA^{I} dimer. Docking of the rRNA fragments from high-resolution crystal structures of ribosome [nucleotides 781 to 865 from H. marismortui (Ban, N., et al., (2000) Science 289, 905-920), 704 to 784 from D. radiodurans (Schliunzen, F., et al., (2001) Nature 413, 814-821), and 685 to 773 from T. thermophilus (Yusupov, M. M., et al., (2001) Science 292, 883-896)] into the RNA-binding cleft of RlmA^{I} showed possible fits of hairpin 35 into the W-shaped RNA-binding cleft; however, hairpin 33 develops steric hindrance with RlmA^{I} when the angle between the hairpins 33 and 35 is small (FIG. 5B). RlmA enzymes do not act on 50S or 70S subunit of ribosome (Hansen, L. H., et al., (2001) J. Mol. Biol. 310, 1001-1110), and it is therefore likely that the modeled (FIG. 5A) 23S rRNA fragment (Mueller, F., et al., (2000) J. Mol. Biol. 298, 35-59) more closely reflects its naked conformation that actually binds to RlmA dimer. The two Zn-fingers of the RlmA dimer apparently interact at the hinges between hairpins 33:35 and 34:35 and consequently define the appropriate shape of the rRNA fragment.

Our current structure and modeling study suggests that most of the RNA:protein interactions in this complex are asymmetric; one monomer interacts differently with the RNA substrate than the other. The asymmetric nature of the RNA:protein interactions may be responsible for the unique fit of the substrate to the enzyme. Docking of the rRNA substrate predicts that regions 6-8, 25, 38-52, 117-119, 138-141, 157-162, and 233-235 of Molecule 1 and 6-8, 25, 38-52, 115-121, and 136-140 of Molecule 2 of the RlmA^{I} dimer are likely to be involved in protein:RNA interactions. The length of the polypeptide linker (amino acids 35-52) between the two domains is 3-4 amino acids shorter in RlmA^{II} than in RlmA^{I} (FIG. 1A). The amino acid sequences of the linker are also distinct for RlmA^{I} and RlmA^{II} classes of the enzymes. This linker region of RlmA enzymes may play a role in precise positioning of G745 (in RlmA^{I}) or G748 (in RlmA^{II}) appropriately with respect to SAM for methylation.

### Example 9

### G745/G748 Methylation

The above analysis suggests that an RlmA dimer is required for binding of its substrate, hairpin 35 of 23S rRNA. However, only one base of the rRNA substrate is methylated and only one RlmA molecule from the dimer is required to catalyze this N1-methylation. In ribosome structures, rRNA hairpin 35 interacts with the large b-sheet of the ribosomal protein L22 and adopts a complementary inverted “U” shape (FIG. 5B), which is different from the unbound structure of the hairpin determined by NMR (Lebars, I., et al., (2003) EMBO J. 22, 183-92). Docking of the L22-bound conformation of hairpin 35 from different ribosome structures (discussed in previous section) shows a reasonable match between the hairpin and the ridge of the W-shaped cleft of RlmA^{I}; in these modeled complexes, two nucleotides (U480 and A844 of H. marismortui (Ban, N., et al., (2000) Science 289, 905-920), U760 and A764 of D. radiodurans (Schlunzen, F., et al., (2001) Nature 413, 814-821), and A747 and A751 of T. thermophilus (Yusupov, M. M., et al., (2001) Science 292, 883-896)), at equivalent E. coli positions 747 and 751, point to two SAM-binding pockets of the RlmA^{I} dimer.

The RlmA^{I} structure, together with sequence comparisons, suggest similar W-shaped conformation of the rRNA binding cleft and mode of binding of the rRNA fragment for both RlmA^{I} and RlmA^{II}. Based on our structure and modeling studies, we speculate that the hairpin 35 adopts a shape complementary to the W-shaped rRNA binding cleft, whether bound to RlmA^{I} or to RlmA^{II}, in which nucleotides G745 and G748 point towards the two SAM-binding sites of the enzyme, as shown schematically in FIG. 5C. Alternatively, hairpin 35 may adopt somewhat different conformations when bound to RlmA^{I} or RlmA^{II}, such that either G745 (FIG. 5A) or G748 is pointed towards one SAM-binding pocket. In these two proposed structures of RlmA:rRNA complex, specific protein:rRNA interactions (e.g., the interactions of the loop connecting the Zn-finger and MTase domains with rRNA hairpin 35) would play decisive role in proper positioning of the correct nucleotide for N1-methylation catalysis.

The crystal structure of E. coli RlmA^{I} has a well-defined and largely positively charged W-shaped RNA-binding cleft formed by asymmetric dimerization (FIG. 4). Structural, functional, and amino acid sequence similarities among RlmA^{I} and RlmA^{II} enzymes (FIG. 1A) suggest a common fold, as well as similar SAM- and RNA-substrate binding modes, for both classes of RlmA enzymes. It appears that the two Zn-binding domains are responsible for recognition and binding of the hairpin 35 region of the 23S rRNA (FIG. 5A). Amino acid sequence comparison of RlmA^{I} and RlmA^{II} and mapping of the conserved region onto the crystal structure of E. coli RlmA^{I} indicate positioning of some of the key conserved amino acid residues at putative RNA-binding regions, at the SAM-binding pocket, and at the dimer interface. Docking the publicly available atomic coordinates for hairpin 35 of 23S rRNA and surrounding regions (Ban, N., et al., (2000) Science 289, 905-920; Schlunzen, F., et al., (2001) Nature 413, 814-821; Mueller, F., et al., (2000) J. Mol. Biol. 298, 35-59; Yusupov, M. M., et al., (2001) Science 292, 883-896; Lebars, I., et al., (2003) EMBO J. 22, 183-92) into the cleft of RlmA^{I} dimer shows complementary RNA:protein structural features. This crystal structure along with earlier reported biochemical data provide a basis for detailed investigations aimed at understanding structural features of the specific recognition of rRNA substrates, the role of a new type of Zn-finger in RNA recognition, general aspects of RNA:protein interactions, and the mechanism of RNA methylation by RlmA enzymes.

### Data Deposition

The atomic coordinates and structure factors for E. coli RlmA^{I} structure have been deposited in the Protein Data Bank (PDB accession code 1P91).

### INDUSTRIAL APPLICABILITY

Compounds identified according to the target and method of this invention would have applications not only in antibacterial therapy, but also in: (a) identification of bacterial RlmA proteins (diagnostics, environmental-monitoring, and sensors applications), (b) labeling of bacterial RlmA proteins (diagnostics, environmental-monitoring, imaging, and sensors applications), (c) immobilization of bacterial RlmA proteins (diagnostics, environmental-monitoring, and sensors applications), (d) purification of bacterial RlmA proteins (biotechnology applications), (e) regulation of bacterial gene expression (biotechnology applications).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated herein by reference.

1. A composition consisting essentially of RlmA protein in crystalline form, wherein the RlmA is RlmA^{I} or RlmA^{II}.

2. A method for screening compound libraries to identify compounds which bind in the cleft of an RlmA protein in crystalline form, wherein said RlmA is RlmA^{I} or RlmA^{II}, and which compounds are potential inhibitors of rRNA-binding or methylation function, comprising X-ray crystallography.

3. The method of claim 2, wherein the compound is soaked into crystal of RlmA or co-crystallized with RlmA molecules in order to identify a compound that binds at the RNA binding site of RlmA.

4. The method of claim 2, comprising detecting interference with the function of RlmA by inhibiting S-adenosylmethionine-binding or other aspects of the catalytic mechanism of the methyltransferase domain.

5. The method of claim 2, comprising identification of the orientations and binding modes of said compounds, for structure-based drug design.

6. A method comprising the use of three-dimensional coordinates of a model for the RlmA:rRNA complex for designing compound libraries for screening.

7. A method of identifying a compound that can be used to treat bacterial infections, either alone or in combination with other antibiotics, comprising identifying a compound for use as an inhibitor of the RlmA, or an rRNA binding domain thereof, and a dataset comprising the three-dimensional coordinates obtained from the RlmA, or an rRNA binding domain thereof.

8. The method of claim 7, wherein the identification of a compound is performed in conjunction with computer modeling.

9. The method of claim 7, further comprising the three-dimensional coordinates of the RlmA and the model of the RlmA:rRNA complex provide methods for (a) designing an inhibitor library for screening, (b) rational optimization of lead compounds, and (c) virtual screening of potential inhibitors.

10. A composition comprising a reaction mixture comprising a complex of a bacterial RlmA protein, or an rRNA binding fragment thereof, and an rRNA fragment that binds said protein.

11. The composition of claim 10, comprising an rRNA binding domain of said RlmA protein.

12. The composition of claim 10, wherein the rRNA binding domain is rRNAhp35.

13. The composition of claim 10, further comprising a compound being tested for inhibitory activity against a bacterial strain.

14. The composition of claim 10, wherein the bacterial RlmA protein or the rRNA is detectably labeled.

15. A method of identifying compounds having inhibitory activity against a bacterial strain, or an ability to interfere with the interaction between RlmA and the rRNA in vitro, comprising:
a) preparing a reaction system comprising a bacterial RlmA protein or a rRNA binding domain thereof, a rRNA that binds said protein or binding domain thereof, and a candidate compound; and
b) detecting extent of binding between the bacterial RlmA protein and the rRNA, wherein reduced binding between the bacterial RlmA protein and the rRNA in the presence of the compound relative to a control is indicative of inhibitory activity of the compound against the bacterial strain or the ability of the compound to interfere with the interaction between RlmA and the rRNA.

16. The method of claim 15, wherein the compounds have inhibitory activity against RNA-binding and S-adenosylmethionine binding in a bacterial strain, and wherein further the compounds can be linked by a flexible linker group to design more effective inhibitors.

17. The method of claim 15, wherein the bacterial RlmA protein or rRNA binding domain thereof is immobilized on a solid support.

18. The method of claim 15, wherein the candidate compound is added to the reaction system prior to or simultaneously with the bacterial RlmA protein and the rRNA.

19. The method of claim 15, wherein the candidate compound is added to the reaction system subsequent to addition of the bacterial RlmA protein and the rRNA.

20. The method of claim 15, further comprising labeling the rRNA, rRNA fragment, bacterial RlmA protein or rRNA binding domain thereof with a detectable label, prior to said detecting.

21. The method of claim 15, wherein the method of identifying compounds having inhibitory activity is selected from the group consisting of (a) NMR chemical shift perturbation of the RlmA protein, (b) gel filtration chromatography, c) sedimentation equilibrium measurements using an analytical ultracentrifuge, (d) sedimentation velocity measurements, (e) amide hydrogen-deuterium exchange measurements using NMR or mass spectrometry, (e) static light scattering measurements, (f) dynamic light scattering measurements, or (g) virtual screening using the structure of the RlmA protein and the model of the RlmA protein-rRNA complex.

22. The method of claim 15, wherein the method of identifying compounds having inhibitory activity comprises NMR chemical shift perturbation measurements conducted in the absence of resonance assignments to detect RlmA-rRNA interactions, wherein the compounds are identified which prevent these interactions.

23. The method of claim 20, wherein the detectable label comprises an antibody or fragment thereof that binds the bacterial RlmA protein or rRNA binding domain thereof.

24. The method of claim 20, wherein the detectable label comprises an enzyme and the reaction system further comprises a substrate for the enzyme.

25. The method of claim 20, wherein the detectable label comprises a radioisotope.

26. The method of claim 20, wherein the detectable label comprises a fluorescent label.

27. The method of claim 20, wherein said detecting is conducted via fluorescent resonance energy transfer.

28. The method of claim 20, wherein said detecting is conducted via fluorescence polarization anisotropy measurements.

29. The method of claim 15, wherein the bacterial RlmA protein or rRNA binding fragment thereof is present in a reaction system selected from the group consisting of (a) a fusion protein with glutathione-5-transferase assay, (b) a fluorescence-detected hpt screening assay, (c) a virtual screening assay using a three-dimensional structure of E coli RlmA^{I}, models of RlmA^{I} or RlmA^{II} proteins and/or models of RlmA-rRNA complexes based on the coordinates of E coli RlmAI, and (d) a high throughput screening assay using a library that is biased based on the three-dimensional structure of E coli RlmA^{I}, models of RlmA^{I} or RlmA^{II} proteins and/or models of RlmA-rRNA complexes based on the coordinates of E coli RlmAI.

30. The method of claim 15, wherein the method of identification comprises a high throughput screening assay.

31. A method of preparing a composition for inhibiting replication of a bacterial strain in vitro or in vivo, comprising:
a) preparing a reaction system comprising a bacterial RlmA protein or a rRNA binding domain thereof, a rRNA that binds said protein or binding domain thereof, and a candidate compound;
b) detecting extent of binding between the bacterial RlmA protein and the rRNA, wherein reduced binding between the bacterial RlmA protein and the rRNA in the presence of the compound relative to a control is indicative of inhibitory activity of the compound against the bacterial strain; and
c) determining extent of a compound identified in b) as having activity to inhibit growth of a bacterial strain in vitro, either alone or in the presence of another antibiotic.

32. The method of claim 31, wherein the RlmA protein is an RlmA protein or an rRNA binding domain thereof.

33. The method of claim 31, wherein the rRNA is rRNAhp35 or other RNA fragments.

34. The method of claim 31, wherein the co-antibiotic is a macrolide antibiotic.

35. A method for identifying a compound that binds to a bacterial RlmA protein in a first entity, comprising the steps of: (a) preparing a reaction solution including the compound to be tested and a first entity including a bacterial RlmA protein; and (b) detecting at least one of the presence, extent, concentration-dependence, or kinetics of binding of the compound to the bacterial RlmA protein.

36. The method of claim 35 wherein the first entity is an intact bacterial RlmA.

37. The method of claim 35 wherein the first entity is a fragment of a bacterial RlmA.

38. The method of claim 35 wherein the first entity is Escherichia coli RlmA or a derivative thereof.

39. The method of claim 35 further comprising the step of: detecting at least one of the presence, extent, concentration-dependence, or kinetics of binding of the compound to a second entity that contains a derivative of a bacterial RlmA protein having at least one amino acid substitution, insertion, or deletion.

40. The method of claim 39 wherein the second entity is a derivative of an intact bacterial RlmA.

41. The method of claim 39 wherein the second entity is a derivative of a fragment of a bacterial RlmA.

42. The method of claim 39 wherein the second entity is a derivative of Escherichia coli RlmA.

43. A method of designing compounds which bind to the rRNA-binding pocket of RlmA, comprising modifying rRNAhp35 to synthesize analogs thereof.

44. A method for identifying a compound that inhibits an activity of a bacterial RlmA by binding to a bacterial RlmA protein, comprising: (a) preparing a reaction solution comprising the compound to be tested and a first entity containing a bacterial RlmA protein; and (b) detecting at least one of the presence, extent, concentration-dependence, or kinetics of inhibition of an activity of said first entity, wherein inhibition involves binding of the compound to the bacterial RlmA.

45. The method of claim 44, wherein inhibition involves binding of the compound to rRNA or rRNA fragments thereof.

46. The method of claim 44 wherein the first entity is an intact bacterial RlmA.

47. The method of claim 44 wherein the first entity is a fragment of a bacterial RlmA.

48. The method of claim 44 wherein first entity is Escherichia coli RlmA or a derivative thereof.

49. The method of claim 44 wherein the activity is RNA binding.

50. The method of claim 44 further comprising the step of: detecting at least one of the presence, extent, concentration-dependence, or kinetics of the inhibition by the compound of the activity of a second entity that contains a derivative of a bacterial RlmA protein sequence having at least one amino-acid substitution, insertion, or deletion.

51. The method of claim 50 wherein the second entity is a derivative of an intact bacterial RlmA A.

52. The method of claim 50 wherein the second entity is a derivative of a fragment of a bacterial RlmA.

53. The method of claim 50 wherein the second entity is Escherichia coli RlmA or a derivative thereof.

54. The method of claim 50 wherein the activity is RNA binding.

55. The method of claim 50 wherein inhibition of an activity of the first entity and inhibition of an activity of the second entity are assessed sequentially.

56. The method of claim 50 wherein inhibition of an activity of the first entity and inhibition of an activity of the second entity are assessed simultaneously.

57. The method of claim 50 wherein at least one of the presence, extent, concentration-dependence, or kinetics of inhibition by the compound of an activity of the first entity also is compared to at least one of the presence, extent, concentration-dependence, or kinetics of inhibition by an inhibitory compound specific to the bacterial RlmA protein of an activity of the second entity.

58. A method for identifying a compound that binds to a bacterial RlmA protein, comprising (a) preparing a reaction solution comprising the compound to be tested, a first entity containing a bacterial RlmA protein, and containing a detectable group within RlmA protein; and (b) detecting a change in a property of the detectable group within RlmA protein.

59. The method of claim 58 wherein the first entity is an intact bacterial RlmA protein.

60. The method of claim 58 wherein the first entity is a fragment of an RlmA protein.

61. The method of claim 58 wherein the first entity is Escherichia coli RlmA or a derivative thereof.

62. The method of claim 58 wherein the compound to be tested contains a chromophore.

63. The method of claim 58 wherein the detectable group contains a fluorophore.

64. The method of claim 58 further comprising measurement of fluorescence resonance energy transfer.

65. The method of claim 58 wherein the detectable group is an NMR-active ¹H, ¹³C or ¹⁵N nucleus.

66. The method of claim 58 further comprising the step of: detecting at least one of the presence, extent, concentration-dependence, or kinetics of the binding of the compound to a second entity that contains a derivative of a bacterial RlmA protein having at least one amino-acid substitution, insertion, or deletion.

67. The method of claim 66 wherein the second entity is a derivative of an intact bacterial RlmA protein.

68. The method of claim 66 wherein the second entity is a derivative of a fragment of a bacterial RlmA protein.

69. The method of claim 66 wherein the second entity is Escherichia coli RlmA or a derivative thereof.

70. A method for identifying a compound that binds to a bacterial RlmA protein, comprising (a) preparing a reaction solution comprising the compound to be tested, a first entity containing a bacterial RlmA protein, a second entity containing an rRNA, or rRNA fragment or variant thereof, specific to the rRNA-binding pocket of the RlmA protein, and containing a detectable group within the rRNA; and (b) detecting a change in a property of the detectable group within rRNA.

71. The method of claim 71, wherein the first entity is an intact bacterial RlmA protein.

72. The method of claim 71, wherein the first entity is a fragment of an RlmA protein.

73. The method of claim 71 wherein the first entity is Escherichia coli RlmA or a derivative thereof.

74. The method of claim 71 wherein the compound to be tested contains a chromophore.

75. The method of claim 71 wherein the detectable group contains a fluorophore.

76. The method of claim 71 wherein the detectable group is an NMR-active ¹H, ¹³C or ¹⁵N nucleus.

77. The method of claim 71 further comprising an assay selected from the group consisting of a measurement of fluorescence resonance energy transfer, changes in absorbance, steady-state fluorescence, time-resolved fluorescence, fluorescence polarization anisotropy, or NMR spectroscopy.

78. A method for identifying a compound that binds to a bacterial RlmA protein, comprising (a) preparing a reaction solution comprising the compound to be tested, a reference compound that binds to a bacterial RlmA protein and a first entity containing a bacterial RlmA protein, and (b) detecting at least one of the presence, extent, concentration-dependence, or kinetics of competition by the compound for binding of the reference compound to the RlmA protein.

79. The method of claim 78 wherein the first entity is an intact bacterial RlmA protein.

80. The method of claim 78 wherein the first entity is a fragment of a bacterial RlmA protein.

81. The method of claim 78 wherein the first entity is Escherichia coli RlmA protein or a derivative thereof.

82. The method of claim wherein the reference compound is selected from the group consisting of a rRNA, a fragment thereof, a variant thereof or rRNAhp35.

83. The method of claim 78 wherein the reference compound contains a detectable group.

84. The method of claim 78 wherein the detectable group contains a chromophore.

85. The method of claim 78 wherein the detectable group contains a fluorophore.

86. The method of claim 78 wherein the reference compound is a chromophore-labeled inhibitory compound specific to the bacterial RlmA protein.

87. The method of claim 78 wherein the reference compound is a fluorophore-labeled inhibitory compound specific to the bacterial RlmA protein.

88. The method of claim 78 further comprising measurement of fluorescence resonance energy transfer.

89. The method of claim 78 wherein displacement of the reference compound is assessed by NMR spectroscopy.

89. The method of claim 78 further comprising the step of: detecting at least one of the presence, extent, concentration-dependence, or kinetics of the binding of the compound to a second entity that contains a derivative of a bacterial RlmA protein having at least one amino acid substitution, insertion, or deletion.

90. The method of claim 78 wherein the second entity is a derivative of an intact bacterial RlmA protein.

91. The method of claim 78 wherein the second entity is a derivative of a fragment of a bacterial RlmA protein.

92. The method of claim 78 wherein the second entity is Escherichia coli RlmA protein or a derivative thereof.

93. The method of claim 78 wherein at least one of the presence, extent, concentration-dependence, or kinetics of binding of the compound to the first entity is compared to at least one of the presence, extent, concentration-dependence, or kinetics of binding of an inhibitory compound specific to the bacterial RlmA protein to the second entity.
